(19) United States
(12) Patent Application Publication
(10) Pub. No.: US 2026/0204414 A1
(43) Pub. Date: Jul. 16, 2026

(54) MACHINE LEARNING MODELS FOR DETECTING OUTLIERS AND ERRONEOUS SENSOR USE CONDITIONS AND CORRECTING, BLANKING, OR TERMINATING GLUCOSE SENSORS

(71) Applicant: Medtronic MiniMed, Inc., Northridge, CA (US)

(72) Inventors: Elaine GEE, Windsor, CA (US); Jeffrey NISHIDA, Redwood City, CA (US); Peter AJEMBA, Canyon Country, CA (US); Keith G. NOGUEIRA, Mission Hills, CA (US); Andrea VARSAVSKY, Santa Monica, CA (US); Georgios MALLAS, Los Angeles, CA (US); John MOON, Northridge, CA (US); Bahram B. NOTGHI, Encino, CA (US)

(21) Appl. No.: 19/558,080

(22) Filed: Mar. 5, 2026

Related U.S. Application Data
(63) Continuation-in-part of application No. 18/324,820, filed on May 26, 2023.
Application No. 18/324,820 is a continuation of application No. 14/980,293, filed on Dec. 28, 2015, now abandoned.
(63) Continuation-in-part of application No. 17/163,186, filed on Jan. 29, 2021, now Pat. No. 12,626,819.
Application No. 17/163,186 is a continuation of application No. 17/121,624, filed on Dec. 14, 2020, now abandoned.

(30) Foreign Application Priority Data
Feb. 20, 2026 (GR) 20260100169

(51) Int. Cl. G16H 50/20; A61B 5/00; A61B 5/145; A61B 5/1473; A61B 5/1495; G06N 3/126; G06N 20/00; G16H 40/40

(52) U.S. Cl. CPC G16H 50/20; A61B 5/14532; A61B 5/14735; A61B 5/1495; G06N 3/126; G06N 20/00; G16H 40/40; A61B 5/6849

(57) ABSTRACT

Techniques disclosed herein relate to sensor data. In some embodiments, the techniques may involve obtaining sensor data. The techniques may further involve applying, to the sensor data, a machine learning model trained to identify outlier measurements. The techniques may further involve determining, based on output from the machine learning model, that the sensor data corresponds to an outlier measurement. The techniques may further involve blanking the sensor data based on the output.

### RELATED APPLICATION DATA

This application claims the benefit of and priority to Greece Application No. 20260100169, filed Feb. 20, 2026 and titled “MACHINE LEARNING MODELS FOR DETECTING OUTLIERS AND ERRONEOUS SENSOR USE CONDITIONS AND CORRECTING, BLANKING, OR TERMINATING GLUCOSE SENSORS.” This application is a continuation-in-part of U.S. patent application Ser. No. 17/163,186, filed Jan. 29, 2021 and titled “MACHINE LEARNING MODELS FOR DETECTING OUTLIERS AND ERRONEOUS SENSOR USE CONDITIONS AND CORRECTING, BLANKING, OR TERMINATING GLUCOSE SENSORS,” which is a continuation of U.S. patent application Ser. No. 17/121,624, filed Dec. 14, 2020 and titled “MACHINE LEARNING MODELS FOR DETECTING OUTLIERS AND ERRONEOUS SENSOR USE CONDITIONS AND CORRECTING, BLANKING, OR TERMINATING GLUCOSE SENSORS.” This application is also a continuation-in-part of U.S. patent application Ser. No. 18/324,820, filed May 26, 2023 and titled “RETROSPECTIVE SENSOR SYSTEMS, DEVICES, AND METHODS,” which is a continuation of U.S. patent application Ser. No. 14/980,293, filed Dec. 28, 2015, and titled “RETROSPECTIVE SENSOR SYSTEMS, DEVICES, AND METHODS.” Each of these applications is incorporated herein by reference in its entirety.

### FIELD

The present technology is generally related to sensor technology, including sensors used for sensing a variety of physiological parameters, e.g., glucose concentration.

### BACKGROUND

Over the years, a variety of sensors have been developed for detecting and/or quantifying specific agents or compositions in a patient's blood, which enable patients and medical personnel to monitor physiological conditions within the patient's body. Illustratively, subjects may wish to monitor blood glucose levels in a subject's body on a continuing basis. Thus, glucose sensors have been developed for use in obtaining an indication of blood glucose levels in a diabetic patient. Such readings are useful in monitoring and/or adjusting a treatment regimen which typically includes the regular administration of insulin to the patient. Presently, a patient can measure his/her blood glucose (“BG”) using a BG measurement device (i.e., glucose meter), such as a test strip meter, a continuous glucose measurement system (or a continuous glucose monitor), or a hospital BG test. BG measurement devices use various methods to measure the BG level of a patient, such as a sample of the patient's blood, a sensor in contact with a bodily fluid, an optical sensor, an enzymatic sensor, or a fluorescent sensor. When the BG measurement device has generated a BG measurement, the measurement is displayed on the BG measurement device.

### SUMMARY

Techniques disclosed herein relate to sensor data. The techniques may be practiced with a processor-implemented method, a system comprising one or more processors and one or more processor-readable media, and/or one or more non-transitory processor-readable media.

In some embodiments, the techniques may involve obtaining sensor data. The techniques may further involve applying, to the sensor data, a machine learning model trained to identify outlier measurements. The techniques may further involve determining, based on output from the machine learning model, that the sensor data corresponds to an outlier measurement. The techniques may further involve blanking the sensor data based on the output.

In some embodiments, the techniques may involve obtaining sensor data. The techniques may further involve applying, to the sensor data, a first machine learning model trained to identify outlier measurements. The techniques may further involve determining, based on output from the machine learning model, that the sensor data corresponds to an outlier measurement. The techniques may further involve in response to determining that the sensor data corresponds to the outlier measurement, applying, to the sensor data, a second machine learning model trained to identify whether the outlier measurement can be compensated. The techniques may further involve determining, based on output from the second machine learning model, that the outlier measurement can be compensated. The techniques may further involve in response to determining that the outlier measurement can be compensated, applying a compensation to the sensor data to generate compensated sensor data.

Various other aspects, features, and advantages will be apparent through the detailed description and the drawings attached hereto. It is also to be understood that both the foregoing general description and the following detailed description are examples and not restrictive of the scope of the invention. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification “a portion,” refers to a sub-part of, or the entirety of, a given item (e.g., data) unless the context clearly dictates otherwise.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A detailed description of embodiments of the invention will be made with reference to the accompanying drawings, wherein like numerals designate corresponding parts in the figures.

FIG. 1 illustrates wearable sensor electronics devices, in accordance with one or more embodiments.

FIG. 2 is a perspective view of a subcutaneous sensor insertion set and block diagram of a sensor electronics device, in accordance with one or more embodiments.

FIG. 3 illustrates a substrate having two sides, a first side which contains an electrode configuration and a second side which contains electronic circuitry, in accordance with one or more embodiments.

FIG. 4 illustrates a block diagram of a sensor electronics device and a sensor including a plurality of electrodes, in accordance with one or more embodiments.

FIG. 5 illustrates an alternative embodiment of the invention including a sensor and a sensor electronics device, in accordance with one or more embodiments.

FIG. 6 illustrates an electronic block diagram of the sensor electrodes and a voltage being applied to the sensor electrodes, in accordance with one or more embodiments.

FIG. 7 shows a flowchart of the steps involved in applying machine learning models to improve integrated continuous glucose monitoring (“iCGM”) performance of continuous glucose monitoring (“CGM”) calibration algorithms, in accordance with one or more embodiments.

FIG. 8 shows a flowchart of the steps involved in applying machine learning models to detect and correct for erroneous sensor use conditions, in accordance with one or more embodiments.

FIG. 9 shows a machine learning model system for making predictions that facilitate both classification of outlier measurements based on iCGM criteria and detection and correction of erroneous sensor use conditions, in accordance with one or more embodiments.

FIG. 10 shows a flow diagram for conditional blanking and termination and error detection and correction, in accordance with one or more embodiments.

FIG. 11 shows a schematic of a sensor feature generator of FIG. 10, in accordance with one or more embodiments.

FIG. 12 shows a graph highlighting error patterns in sensor data, in accordance with one or more embodiments.

FIG. 13 shows a flowchart of an example process for compensating outlier sensor measurements in accordance with one or more embodiments.

### DETAILED DESCRIPTION

In the following description, reference is made to the accompanying drawings which form a part hereof and which illustrate several embodiments of the present inventions. It is understood that other embodiments may be utilized, and structural and operational changes may be made without departing from the scope of the present inventions.

The inventions herein are described below with reference to flowchart illustrations of methods, systems, devices, apparatus, and programming and computer program products. It will be understood that each block of the flowchart illustrations, and combinations of blocks in the flowchart illustrations, can be implemented by programing instructions, including computer program instructions (as can any menu screens described in the figures). These computer program instructions may be loaded onto a computer or other programmable data processing apparatus (such as a controller, microcontroller, or processor in a sensor electronics device) to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks, and/or menus presented herein. Programming instructions may also be stored in and/or implemented via electronic circuitry (e.g., storage circuitry, processing circuitry), including integrated circuits (ICs) and Application Specific Integrated Circuits (ASICs) used in conjunction with sensor devices, apparatuses, and systems. The following terms and definitions may also be used herein:

| Term | Definition |
|---|---|
| BG | Blood Glucose value |
| | in mg/dL typically |
| | from a fingerstick |
| | reading. Assumed use is |
| | for a sensor calibration |
| Calibrated Mode | Sensor operation mode |
| | in which the algorithm |
| | expects to receive BG |
| | calibrations as part of |
| | regular operation |
| CE | Calibration Error |
| CF (or calFactor) | Calibration Factor, |
| | sensor sensitivity to |
| | glucose used to calculate |
| | sensor glucose. Units |
| | are mg/dL/nA |
| CR (or cr) | Calibration Ratio, |
| | sensitivity based on a |
| | single BG and Isig |
| Discard | Packet flagged to be |
| | invalid based on Isig. |
| early calibration | Temporary CF update on |
| | the packet following a BG |
| EIS | Electrochemical Impedance |
| | Spectroscopy, Diagnostic |
| | capability to measure |
| | impedances at varying |
| | frequencies applied by |
| | the AFE IC |
| final calibration | Refers to updates to CF |
| | and other variables which |
| | may occur 10-15 minutes |
| | after a BG entry |
| fisig | Filtered Isig, used in |
| | calibration and SG |
| | calculation |
| GST | Glucose Sensor Transmitter |
| GOx | Glucose Oxidase |
| initialization | Sensor Initialization. This |
| | typically refers to data |
| | collection activities during |
| | sensor warm up period |
| Instant calibration error | CE check based on prior Isig, |
| | determines if a BG can be |
| | used for calibration |
| invalid packet | Refers to a packet being |
| | flagged as invalid. Packets |
| | flagged as invalid do not |
| | show SG to the user. |
| Isig | 5-minute reading of sensor |
| | current in nA. Sometimes |
| | called “raw Isig” |
| Isig1 | 1-minute reading of sensor |
| | current in nA. Sometimes |
| | called “1-minute Isig” |
| Isig Dip | Isig Dip Calibration. Refers |
| | to logic which may adjust CF |
| | following a calibration on an |
| | abnormally low Isig |
| MAX_CR | Maximum acceptable CR |
| MIN_CR | Minimum acceptable CR |
| Not Calibrated Mode | Sensor operation mode in |
| | which the algorithm does |
| | not expect to receive BG |
| | calibrations as part of |
| | regular operations. The algorithm |
| | can utilize BG calibrations if |
| | any is made available. |
| Packet (or SG | Refers to the collection of |
| Packet or Isig Packet) | variables calculated at the |
| | 5-minute interval, including |
| | Isig, sg, etc. |
| SG | Sensor Glucose value in mg/dL |
| Vset | Voltage potential |

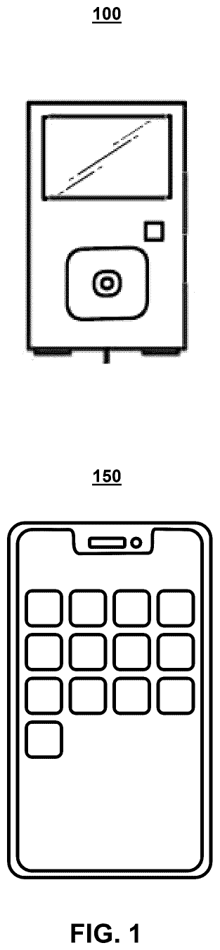

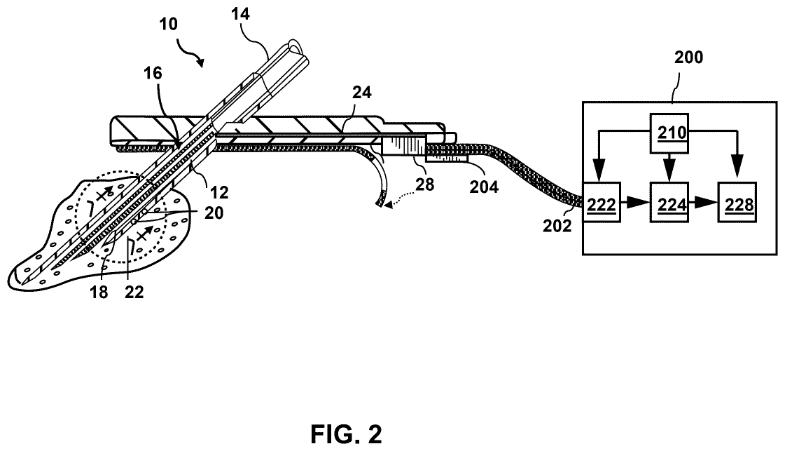

FIG. 1 illustrates wearable sensor electronics devices 100 and 150, in accordance with one or more embodiments. In some embodiments, wearable sensor electronics device 100 may be an infusion pump. In some embodiments, the infusion pump may include a display. In some embodiments, wearable sensor electronics device 100 may be a combination infusion pump/glucose sensor. In some embodiments, wearable sensor electronics device 150 may be a cellular phone or any computing device. In some embodiments, wearable sensor electronics devices 100 and 150 may include a computer, a personal digital assistant, a pager, or any other suitable wearable device. In some embodiments, wearable sensor electronics devices 100 and 150 may house components described below in relation to FIGS. 2-6.

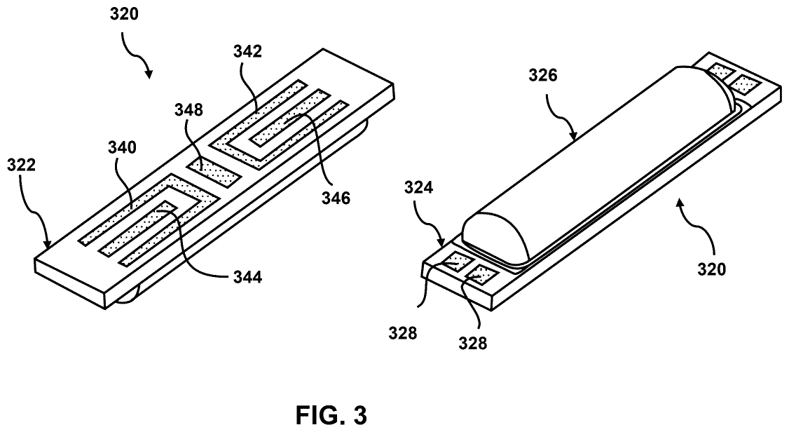

FIG. 2 is a perspective view of a subcutaneous sensor insertion set and a block diagram of a sensor electronics device (e.g., wearable sensor electronics devices 100 or 150, as shown in FIG. 1, or any other suitable sensor electronics device). As illustrated in FIG. 2, a subcutaneous sensor set 10 is provided for subcutaneous placement of an active portion of a flexible sensor 12 (see, e.g., FIG. 3), or the like, at a selected site in the body of a user. The subcutaneous or percutaneous portion of the sensor set 10 includes a hollow, slotted insertion needle 14, and a cannula 16. The needle 14 is used to facilitate quick and easy subcutaneous placement of the cannula 16 at the subcutaneous insertion site. Inside the cannula 16 is a sensing portion 18 of the sensor 12 to expose one or more sensor electrodes 20 to the user's bodily fluids through a window 22 formed in the cannula 16. In one embodiment, the one or more sensor electrodes 20 may include a counter electrode, a reference electrode, and one or more working electrodes. After insertion, the insertion needle 14 is withdrawn to leave the cannula 16 with the sensing portion 18 and the sensor electrodes 20 in place at the selected insertion site.

In particular embodiments, the subcutaneous sensor set 10 facilitates accurate placement of a flexible thin film electrochemical sensor 12 of the type used for monitoring specific blood parameters representative of a user's condition. The sensor 12 monitors glucose levels in the body and may be used in conjunction with automated or semi-automated medication infusion pumps (e.g., wearable sensor electronics device 100, as shown in FIG. 1) of the external or implantable type to control delivery of insulin to a diabetic patient, as described, e.g., in U.S. Pat. No. 4,562,751; 4,678,408; 4,685,903 or 4,573,994, which are herein incorporated by reference.

Particular embodiments of the flexible electrochemical sensor 12 are constructed in accordance with thin film mask techniques to include elongated thin film conductors embedded or encased between layers of a selected insulative material such as polyimide film or sheet, and membranes. The sensor electrodes 20 at a tip end of the sensing portion 18 are exposed through one of the insulative layers for direct contact with patient blood or other body fluids, when the sensing portion 18 (or active portion) of the sensor 12 is subcutaneously placed at an insertion site. The sensing portion 18 is joined to a connection portion 24 that terminates in conductive contact pads, or the like, which are also exposed through one of the insulative layers. In alternative embodiments, other types of implantable sensors, such as chemical based, optical based, or the like, may be used.

As is known in the art, the connection portion 24 and the contact pads are generally adapted for a direct wired electrical connection to a suitable monitor or sensor electronics device 200 (e.g., wearable sensor electronics devices 100 or 150, as shown in FIG. 1, or any other suitable sensor electronics device) for monitoring a user's condition in response to signals derived from the sensor electrodes 20. Further description of flexible thin film sensors of this general type are to be found in U.S. Pat. No. 5,391,250, entitled METHOD OF FABRICATING THIN FILM SENSORS, which is herein incorporated by reference. The connection portion 24 may be conveniently connected electrically to the monitor or sensor electronics device 200 or by a connector block 28 (or the like) as shown and described in U.S. Pat. No. 5,482,473, entitled FLEX CIRCUIT CONNECTOR, which is also herein incorporated by reference. Thus, in accordance with some embodiments, subcutaneous sensor sets 10 may be configured or formed to work with either a wired or a wireless characteristic monitor system.

The sensor electrodes 20 may be used in a variety of sensing applications and may be configured in a variety of ways. For example, the sensor electrodes 20 may be used in physiological parameter sensing applications in which some type of biomolecule is used as a catalytic agent. For example, the sensor electrodes 20 may be used in a glucose and oxygen sensor having a glucose oxidase (GOx) enzyme catalyzing a reaction with the sensor electrodes 20. The sensor electrodes 20, along with a biomolecule or some other catalytic agent, may be placed in a human body in a vascular or non-vascular environment. For example, the sensor electrodes 20 and biomolecule may be placed in a vein and be subjected to a blood stream or may be placed in a subcutaneous or peritoneal region of the human body.

The monitor 200 may also be referred to as a sensor electronics device 200. The monitor 200 may include a power source 210, a sensor interface 222, processing electronics 224, and data formatting electronics 228. The monitor 200 may be coupled to the sensor set 10 by a cable 202 through a connector that is electrically coupled to the connector block 28 of the connection portion 24. In an alternative embodiment, the cable may be omitted. In this embodiment, the monitor 200 may include an appropriate connector for direct connection to the connection portion 204 of the sensor set 10. The sensor set 10 may be modified to have the connector portion 204 positioned at a different location, e.g., on top of the sensor set to facilitate placement of the monitor 200 over the sensor set.

In one embodiment, the sensor interface 222, the processing electronics 224, and the data formatting electronics 228 are formed as separate semiconductor chips, however, alternative embodiments may combine the various semiconductor chips into a single, or multiple customized semiconductor chips. The sensor interface 222 connects with the cable 202 that is connected with the sensor set 10.

The power source 210 may be a battery. The battery can include three series silver oxide 357 battery cells. In alternative embodiments, different battery chemistries may be utilized, such as lithium-based chemistries, alkaline batteries, nickel metal hydride, or the like, and a different number of batteries may be used. The monitor 200 provides power to the sensor set via the power source 210, through the cable 202 and cable connector 204. In one embodiment, the power is a voltage provided to the sensor set 10. In another embodiment, the power is a current provided to the sensor set 10. In an embodiment, the power is a voltage provided at a specific voltage to the sensor set 10.

FIG. 3 illustrates an implantable sensor, and electronics for driving the implantable sensor in accordance with one embodiment. FIG. 3 shows a substrate 320 having two sides, a first side 322 of which contains an electrode configuration and a second side 324 of which contains electronic circuitry (e.g., storage circuitry, processing circuitry, etc.). As may be seen in FIG. 3, a first side 322 of the substrate comprises two counter electrode-working electrode pairs 340, 342, 344, 346 on opposite sides of a reference electrode 348. A second side 324 of the substrate comprises electronic circuitry. As shown, the electronic circuitry may be enclosed in a hermetically sealed casing 326, providing a protective housing for the electronic circuitry. This allows the sensor substrate 320 to be inserted into a vascular environment or other environment which may subject the electronic circuitry to fluids. By sealing the electronic circuitry in a hermetically sealed casing 326, the electronic circuitry may operate without risk of short circuiting by the surrounding fluids. Also shown in FIG. 3 are pads 328 to which the input and output lines of the electronic circuitry may be connected. The electronic circuitry itself may be fabricated in a variety of ways. According to an embodiment, the electronic circuitry may be fabricated as an integrated circuit using techniques common in the industry.

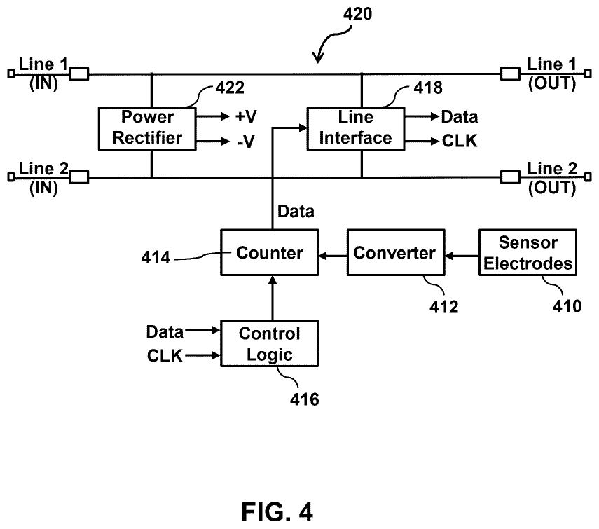

FIG. 4 illustrates a general block diagram of an electronic circuit for sensing an output of a sensor according to one embodiment. At least one pair of sensor electrodes 410 may interface to a data converter 412, the output of which may interface to a counter 414. The counter 414 may be controlled by control logic 416. The output of the counter 414 may connect to a line interface 418. The line interface 418 may be connected to input and output lines 420 and may also connect to the control logic 416. The input and output lines 420 may also be connected to a power rectifier 422.

The sensor electrodes 410 may be used in a variety of sensing applications and may be configured in a variety of ways. For example, the sensor electrodes 410 may be used in physiological parameter sensing applications in which some type of biomolecule is used as a catalytic agent. For example, the sensor electrodes 410 may be used in a glucose and oxygen sensor having a GOx enzyme catalyzing a reaction with the sensor electrodes 410. The sensor electrodes 410, along with a biomolecule or some other catalytic agent, may be placed in a human body in a vascular or non-vascular environment. For example, the sensor electrodes 410 and biomolecule may be placed in a vein and be subjected to a blood stream.

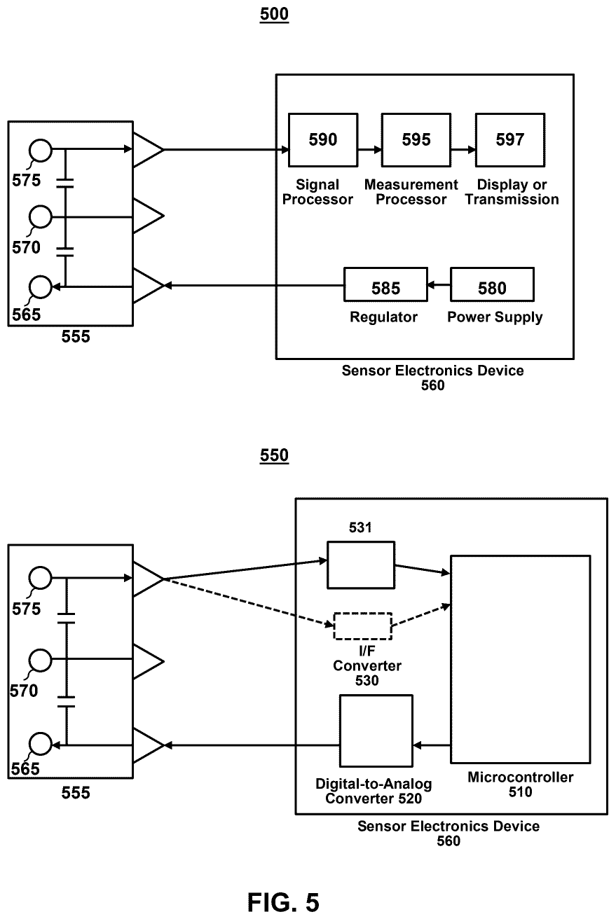

FIG. 5 illustrates a block diagram of a sensor electronics device (e.g., wearable sensor electronics devices 100 or 150, as shown in FIG. 1, or any other suitable sensor electronics device) and a sensor including a plurality of electrodes according to an embodiment herein. FIG. 5 includes system 500. System 500 includes a sensor 555 and a sensor electronics device 560. The sensor 555 includes a counter electrode 565, a reference electrode 570, and a working electrode 575. The sensor electronics device 560 includes a power supply 580, a regulator 585, a signal processor 590, a measurement processor 595, and a display/transmission module 597. The power supply 580 provides power (in the form of either a voltage, a current, or a voltage including a current) to the regulator 585. The regulator 585 transmits a regulated voltage to the sensor 555. In one embodiment, the regulator 585 transmits a voltage to the counter electrode 565 of the sensor 555.

The sensor 555 creates a sensor signal indicative of a concentration of a physiological characteristic being measured. For example, the sensor signal may be indicative of a blood glucose reading. In an embodiment utilizing subcutaneous sensors, the sensor signal may represent a level of hydrogen peroxide in a subject. In an embodiment where blood or cranial sensors are utilized, the amount of oxygen is being measured by the sensor and is represented by the sensor signal. In an embodiment utilizing implantable or long-term sensors, the sensor signal may represent a level of oxygen in the subject. The sensor signal is measured at the working electrode 575. In one embodiment, the sensor signal may be a current measured at the working electrode. In an embodiment, the sensor signal may be a voltage measured at the working electrode.

The signal processor 590 receives the sensor signal (e.g., a measured current or voltage) after the sensor signal is measured at the sensor 555 (e.g., the working electrode). The signal processor 590 processes the sensor signal and generates a processed sensor signal. The measurement processor 595 receives the processed sensor signal and calibrates the processed sensor signal utilizing reference values. In one embodiment, the reference values are stored in a reference memory and provided to the measurement processor 595. The measurement processor 595 generates sensor measurements. The sensor measurements may be stored in a measurement memory (not shown) or by circuitry (e.g., storage circuitry). The sensor measurements may be sent to a display/transmission device to be either displayed on a display in a housing with the sensor electronics or transmitted to an external device.

The sensor electronics device 560 may be a monitor which includes a display to display physiological characteristics readings. The sensor electronics device 560 may also be installed in a desktop computer, a pager, a television including communications capabilities, a laptop computer, a server, a network computer, a personal digital assistant (PDA), a portable telephone including computer functions, an infusion pump including a display (e.g., wearable sensor electronics device 100, as shown in FIG. 1), a glucose sensor including a display, and/or a combination infusion pump/glucose sensor (e.g., wearable sensor electronics device 100, as shown in FIG. 1). The sensor electronics device 560 may be housed in a blackberry (e.g., wearable sensor electronics device 150, as shown in FIG. 1), a network device, a home network device, or an appliance connected to a home network.

FIG. 5 also includes system 550. System 550 includes a sensor electronics device 560 and a sensor 555. The sensor includes a counter electrode 565, a reference electrode 570, and a working electrode 575. The sensor electronics device 560 includes a microcontroller 510 and a digital-to-analog converter (DAC) 520. The sensor electronics device 560 may also include a current-to-frequency converter (I/F converter) 530.

The microcontroller 510 includes software program code, which when executed, or programmable logic which, causes the microcontroller 510 to transmit a signal to the DAC 520, where the signal is representative of a voltage level or value that is to be applied to the sensor 555. The DAC 520 receives the signal and generates the voltage value at the level instructed by the microcontroller 510. In one embodiment, the microcontroller 510 may change the representation of the voltage level in the signal frequently or infrequently. Illustratively, the signal from the microcontroller 510 may instruct the DAC 520 to apply a first voltage value for one second and a second voltage value for two seconds.

The sensor 555 may receive the voltage level or value. In one embodiment, the counter electrode 565 may receive the output of an operational amplifier which has as inputs the reference voltage and the voltage value from the DAC 520. The application of the voltage level causes the sensor 555 to create a sensor signal indicative of a concentration of a physiological characteristic being measured. In an embodiment, the microcontroller 510 may measure the sensor signal (e.g., a current value) from the working electrode. Illustratively, a sensor signal measurement circuit 531 may measure the sensor signal. In an embodiment, the sensor signal measurement circuit 531 may include a resistor and the current may be passed through the resistor to measure the value of the sensor signal. In an embodiment, the sensor signal may be a current level signal and the sensor signal measurement circuit 531 may be a current-to-frequency (I/F) converter 530. The current-to-frequency converter 530 may measure the sensor signal in terms of a current reading, convert it to a frequency-based sensor signal, and transmit the frequency-based sensor signal to the microcontroller 510. In some embodiments, the microcontroller 510 may be able to receive frequency-based sensor signals easier than non-frequency-based sensor signals. The microcontroller 510 receives the sensor signal, whether frequency-based or non-frequency-based, and determines a value for the physiological characteristic of a subject, such as a blood glucose level. The microcontroller 510 may include program code, which when executed or run, is able to receive the sensor signal and convert the sensor signal to a physiological characteristic value. In one embodiment, the microcontroller 510 may convert the sensor signal to a blood glucose level. In an embodiment, the microcontroller 510 may utilize measurements stored within an internal memory or by circuitry (e.g., storage circuitry) in order to determine the blood glucose level of the subject. In an embodiment, the microcontroller 510 may utilize measurements stored within a memory external to the microcontroller 510 or by circuitry to assist in determining the blood glucose level of the subject.

After the physiological characteristic value is determined by the microcontroller 510, the microcontroller 510 may store measurements of the physiological characteristic values for a number of time periods. For example, a blood glucose value may be sent to the microcontroller 510 from the sensor in intervals (e.g., every second or five seconds), and the microcontroller may save sensor measurements in intervals (e.g., for five minutes or ten minutes of estimated SG values from the calibration algorithm). The microcontroller 510 may transfer the measurements of the physiological characteristic values to a display on the sensor electronics device 560 or a display interface of another user device. For example, the sensor electronics device 560 may be a monitor which includes a display that provides a blood glucose reading for a subject. In one embodiment, the microcontroller 510 may transfer the measurements of the physiological characteristic values to an output interface of the microcontroller 510. The output interface of the microcontroller 510 may transfer the measurements of the physiological characteristic values, e.g., sensor glucose values, to an external device, e.g., an infusion pump (e.g., wearable sensor electronics device 100, as shown in FIG. 1), a combined infusion pump/glucose meter (e.g., wearable sensor electronics device 100, as shown in FIG. 1), a computer, a personal digital assistant, a pager, a network appliance, a server, a cellular phone (e.g., wearable sensor electronics device 150, as shown in FIG. 1), or any computing device.

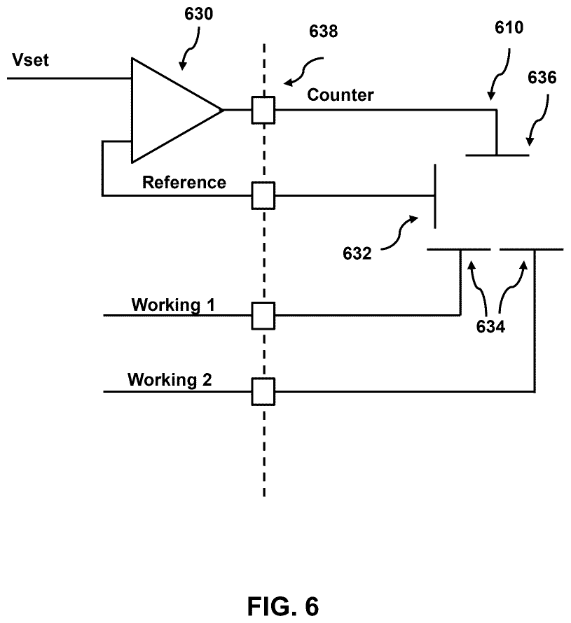

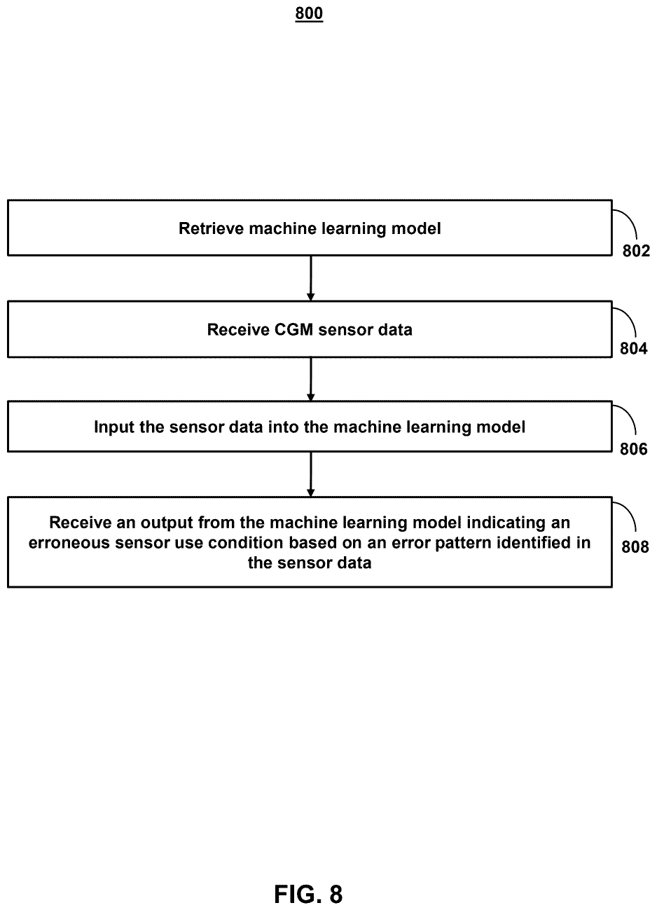

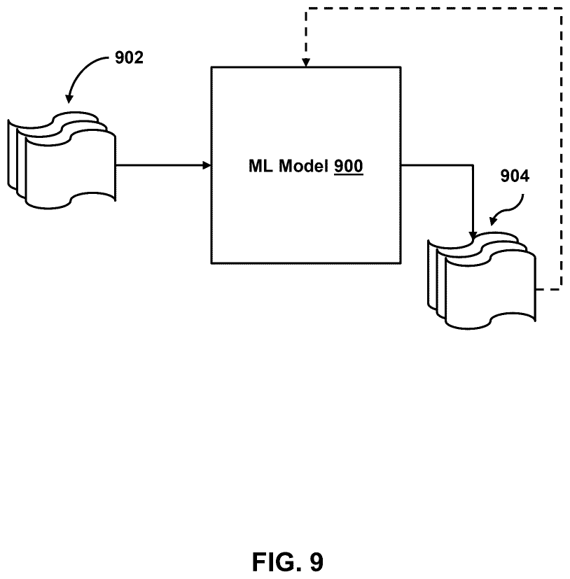

FIG. 6 illustrates an electronic block diagram of the sensor electrodes and a voltage being applied to the sensor electrodes according to an embodiment. In some embodiments, FIG. 6 may illustrate an electrode with a GOx sensor and/or an electrode capable of sensing GOx. For example, FIG. 6 may illustrate a working electrode with a GOx sensor that functions with a background electrode in which the background electrode has no GOx sensor (e.g., as discussed below in relation to FIGS. 8 and 9). The system may then compare the first signal and the second signal to detect ingestion of a medication by the user. The system may generate a sensor glucose value based on the comparison. In the embodiment illustrated in FIG. 6, an op amp 630 or other servo-controlled device may connect to sensor electrodes 610 through a circuit/electrode interface 638. The op amp 630, utilizing feedback through the sensor electrodes, attempts to maintain a prescribed voltage (what the DAC may desire the applied voltage to be) between a reference electrode 632 and a working electrode 634 by adjusting the voltage at a counter electrode 636. Current may then flow from a counter electrode 636 to a working electrode 634. Such current may be measured to ascertain the electrochemical reaction between the sensor electrodes 610 and the biomolecule of a sensor that has been placed in the vicinity of the sensor electrodes 610 and used as a catalyzing agent. The circuitry (e.g., processing circuitry) disclosed in FIG. 6 may be utilized in a long-term or implantable sensor or may be utilized in a short-term or subcutaneous sensor.

In a long-term sensor embodiment, where a GOx enzyme is used as a catalytic agent in a sensor, current may flow from the counter electrode 636 to a working electrode 634 only if there is oxygen in the vicinity of the enzyme and the sensor electrodes 610. Illustratively, if the voltage set at the reference electrode 632 is maintained at about 0.5 volts, the amount of current flowing from the counter electrode 636 to a working electrode 634 has a fairly linear relationship with unity slope to the amount of oxygen present in the area surrounding the enzyme and the electrodes. Thus, increased accuracy in determining an amount of oxygen in the blood may be achieved by maintaining the reference electrode 632 at about 0.5 volts and utilizing this region of the current-voltage curve for varying levels of blood oxygen. Different embodiments may utilize different sensors having biomolecules other than a glucose oxidase enzyme and may, therefore, have voltages other than 0.5 volts set at the reference electrode.

As discussed above, during initial implantation or insertion of the sensor 610, the sensor 610 may provide inaccurate readings due to the adjusting of the subject to the sensor and also electrochemical byproducts caused by the catalyst utilized in the sensor. A stabilization period is needed for many sensors in order for the sensor 610 to provide accurate readings of the physiological parameter of the subject. During the stabilization period, the sensor 610 does not provide accurate blood glucose measurements. Users and manufacturers of the sensors may desire to improve the stabilization timeframe for the sensor so that the sensors can be utilized quickly after insertion into the subject's body or a subcutaneous layer of the subject.

In previous sensor electrode systems, the stabilization period or timeframe was one hour to three hours. In order to decrease the stabilization period or timeframe and increase the timeliness of accuracy of the sensor, a sensor (or electrodes of a sensor) may be subjected to a number of pulses rather than the application of one pulse followed by the application of another voltage, for the second time period. In one embodiment, the first voltage may be 1.07 volts. In an embodiment, the first voltage may be 0.535 volts. In an embodiment, the first voltage may be approximately 0.7 volts.

The techniques disclosed herein may detect outlier measurements in sensor data and may determine, responsive to detecting the outlier, whether the measurements are to be blanked. As described below in more detail, in some embodiments, blanking a sensor measurement may involve inhibiting display of the sensor measurement on a display, transmitting the sensor measurement to a paired device (e.g., a mobile phone, an insulin infusion pump, etc.) with an indication that the sensor measurement is not to be used for calculation of insulin dosages, or the like. In some embodiments, outlier measurements may be detected by providing data associated with the sensor to a trained machine learning model, where the trained machine learning model has been trained to classify a given sensor measurement as an outlier or as not an outlier.

In some embodiments, the input provided to the machine learning model may be multi-dimensional. For example, the input may include multiple data points or features that have been concatenated into a vector, and the vector is provided as the input to the trained machine learning model. Example features may include iSig values, Vctr values, EIS values, manufacturing information, data obtained with the sensor during a calibration process at a manufacturing facility, data obtained from a test sensor at the manufacturing facility where the test sensor is of the same lot or batch as the sensor, etc. Example manufacturing information includes a lot number associated with the sensor, information indicative of a manufacturing process or specification used in manufacturing the sensor, etc. Calibration information may include sampled electrical parameters (e.g., iSig values, Vctr values, EIS values, etc.) obtained from a sensor from the same lot obtained during an in vitro test (e.g., when electrical parameters of a tested sensor are measured under different glucose concentrations in an in vitro test). In one example, a multi-dimensional vector may include one or more in vivo measurements (e.g., one or more iSig values, one or more Vctr values, one or more EIS values, etc.) measured while the sensor is inserted at least partially subcutaneously in the patient, and/or one or more in vitro measurements (e.g., obtained from the sensor, or a sensor manufactured as part of the same batch or lot as the sensor, during a calibration process associated with manufacturing). Note that, in instances in which in vitro information is utilized, the in vitro information may be stored in memory of the sensor. For example, the in vitro information may be stored in the memory of the sensor as part of the manufacturing process of the sensor, e.g., after measurement of the in vitro information. As discussed above, such in vitro information may include in vitro measurements measured by the sensor itself, or with a test sensor from the same lot or batch as the sensor (and therefore, presumably having similar in vitro characteristics as the sensor).

By utilizing multiple features, the machine learning model may consider both current data from the sensor while being used in vivo by the patient, as well as data indicating a typical behavior of the sensor. Accordingly, the machine learning model may be trained to identify anomalous sensor behavior that is anomalous for a particular sensor, given its manufacturing conditions. By way of example, in vitro data may indicate that a particular sensor tends to have higher iSig values compared to other sensors. Accordingly, by including in vitro data (e.g., in vitro iSig data) as part of a multi-dimensional feature vector, as well as in vivo iSig data, a trained machine learning model may be able to identify whether relatively high in vivo iSig data is anomalous high (and therefore, is an outlier) given the in vitro behavior of the sensor. This may allow more accurate detections of outlier sensor measurements, both by avoiding false positives of outlier measurements (where measurements are erroneously flagged as outliers), as well as avoiding false negatives (where outlier measurements are not identified). Avoiding false positives allows more sensor data to be used (by not blanking sensor data that is not an outlier), and avoids termination of a sensor that is not producing outlier measurements. Avoiding false negatives improves medical treatment by increasing the accuracy of sensor measurements used to determined insulin dosages.

It should be understood that each feature utilized as part of a multi-dimensional vector may itself be a vector, or series of values. For example, in an instance in which iSig is a feature included in a multi-dimensional vector that is an input to a machine learning model, iSig may be represented as a time series of multiple iSig measurements (e.g., five measurements, ten measurements, twenty measurements, etc.).

In some embodiments, a trained machine learning model that has been trained to identify outlier measurements (e.g., in order to determine whether a particular sensor measurement is to be blanked) may be trained using integrated continuous glucose monitoring (iCGM) criteria. Conventional techniques have used traditional error reduction techniques to improve the standard error of predictions by the machine learning model. In contrast, the techniques disclosed herein train a model to meet iCGM criteria. For example, the model may be trained using a loss function that prioritizes identifying outlier measurements subject to iCGM criteria. In general, iCGM criteria refer to criteria that specify accuracy of CGM devices. Such criteria include accuracy criteria (e.g., that >87% of readings must be within +/−20% of blood glucose values for levels >=70 mg/dL and within +/−15 mg/dL for levels <70 mg/dL), that hypoglycemic and hyperglycemic readings cannot be misaligned (e.g., that the CGM cannot give readings of >180 mg/dL when the true blood glucose level is <70 mg/dL), and the like. By training the model subject to iCGM criteria rather than general error metrics, the machine learning model is trained to identify outliers in a manner that is consistent with accuracy and safety standards across devices.

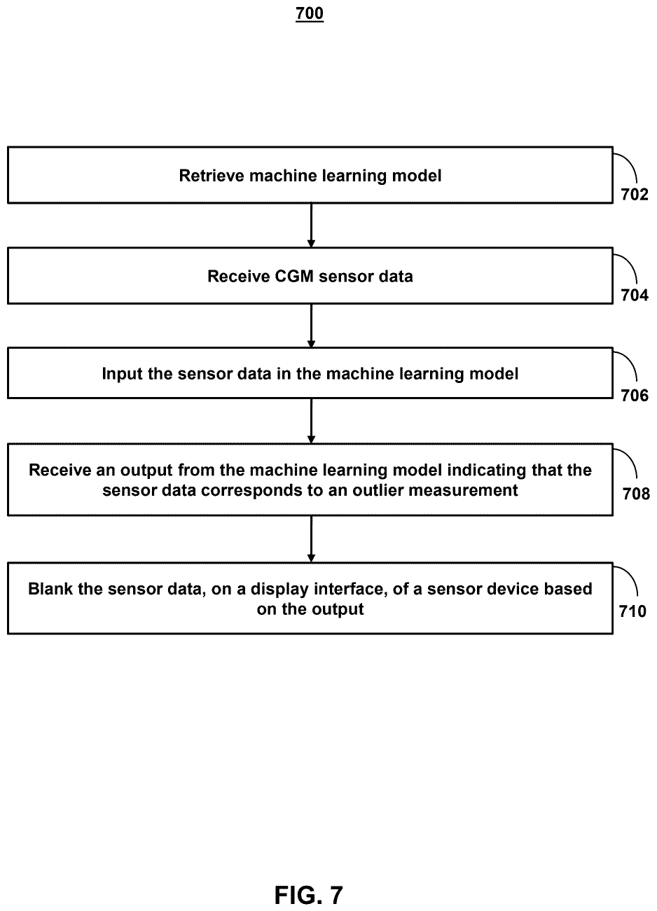

FIG. 7 shows a flowchart of the steps involved in applying machine learning models (e.g., supervised machine learning models, unsupervised machine learning models, semi-supervised machine learning models, or any other suitable type of machine learning model) to improve integrated continuous glucose monitoring (“iCGM”) performance of continuous glucose monitoring (“CGM”) calibration algorithms by classifying outlier measurements based on iCGM criteria, in accordance with one or more embodiments. For example, process 700 may represent the steps taken by one or more devices as shown in FIGS. 1-6.

At step 702, process 700 (e.g., using components described in FIGS. 1-6) retrieves a machine learning model. In some embodiments, the machine learning model is trained to identify outlier measurements based on iCGM criteria using training data. For example, the training data may comprise clinical data on iCGM performance. As discussed below in relation to FIG. 7, process 700 may classify the training data in a number of ways. For example, in some embodiments, process 700 may determine whether a set of training sensor data in the training data corresponds to known classification, wherein the known classification comprises a large negative bias, large positive bias, or nominal accuracy. In some embodiments, in order to determine whether the training sensor data has a large negative bias, large positive bias, or nominal accuracy, process 700 may compare the training sensor data to one or more thresholds (e.g., a negative bias threshold or a positive bias threshold). In some embodiments, this determination may be calibration algorithm dependent since the bias is calculated from the sensor glucose value. Based on whether the training sensor data satisfies the negative bias threshold, process 700 may determine whether the training sensor data has outlier performance. Based on whether the training sensor data satisfies the positive bias threshold, process 700 may determine whether the training sensor data has a large positive bias. If the training sensor data does not satisfy either threshold, process 700 may determine that the training sensor data has nominal accuracy. It should be noted that iCGM criteria are calculated on aggregate data. While it may be possible to calculate bias based on the estimated SG values (e.g., predicted from the sensor data), the machine learning model can predict signals that have outlier behavior that can skew iCGM performance downstream (e.g., as calculated based on the aggregate data).

In some other embodiments, process 700 may determine whether a set of training sensor data in the training data corresponds to known classification, wherein the known classification comprises poor accuracy, intermediate accuracy, or good accuracy. In some embodiments, accuracy may be measured using the mean absolute relative difference (MARD) and bias. Depending on the range of the estimated SG value, a hit criteria for iCGM may shift between the MARD and bias metrics. For example, for certain SG values, the hit criteria may specify a certain bias, while for other SG values, the hit criteria may specify a MARD. In some embodiments, in order to determine whether the training sensor data has poor accuracy, intermediate accuracy, or good accuracy, process 700 may compare the training sensor data to one or more thresholds (e.g., an intermediate accuracy threshold and a good accuracy threshold). Based on whether the training sensor data satisfies the intermediate accuracy threshold, process 700 may determine whether the training sensor data has intermediate accuracy. Based on whether the training sensor data satisfies the good accuracy threshold, process 700 may determine whether the training sensor data has good accuracy. If the training sensor data does not satisfy either threshold, process 700 may determine that the training sensor data has poor accuracy. In some embodiments, process 700 may label the training sensor data with the known classification.

At step 704, process 700 (e.g., using components described in FIGS. 1-6) receives CGM sensor data. For example, process 700 may receive the sensor data at the sensor device. For example, the sensor data may comprise an Interstitial Current Signal (“Isig”), counter voltage (“Vcntr”), and Electrochemical Impedance Spectroscopy Signal (“EIS”). In some embodiments, process 700 may receive the sensor data in first time intervals (e.g., every five minutes or any other predetermined period of time).

At step 706, process 700 (e.g., using components described in FIGS. 1-6) inputs the sensor data in the machine learning model. For example, inputting the sensor data in the machine learning model may comprise generating a multi-dimensional feature input based on the sensor data. In some embodiments, the multi-dimensional feature input may include Isig, Isig rate of change, Isig Noise, real and imaginary EIS signals at a range of frequencies, sensor age (e.g., time since initialization), Vcntr, or other features.

At step 708, process 700 (e.g., using components described in FIGS. 1-6) receives an output from the machine learning model indicating that the sensor data corresponds to an outlier measurement. In some embodiments, the machine learning model may indicate a known classification to which the sensor data corresponds (e.g., large negative bias, large positive bias, nominal accuracy, poor accuracy, intermediate accuracy, good accuracy, etc.).

At step 710, process 700 (e.g., using components described in FIGS. 1-6) blanks the sensor data based on the output. For example, blanking may comprise temporarily removing, blocking, replacing, freezing, or otherwise blanking the sensor data (e.g., eschew transmitting sensor data to another device, eschew displaying sensor data on a display interface). Note that, in some embodiments, blanking sensor data may involve transmitting sensor data to a paired device in connection with an indication that the sensor data is anomalous and/or is not to be used (e.g., is not to be displayed, is not to be used for calculating insulin dosages, etc.) In some embodiments, blanking outlier signals may limit the number of poor sensor glucose estimates that reach the user, which could lead to adverse treatment decisions. For example, reporting a sensor glucose value that is over-reading when a user is in a hypoglycemic state could lead to incorrect treatment decisions, such as insulin dosing that further drives the user deeper into hypoglycemia. Blanking outliers can reduce the instances in which sensor performance falls into the high-risk zones of the Clarke Error Grid (e.g., which quantifies clinical accuracy of patient estimates of their current blood glucose as compared to the blood glucose value obtained in their meter) and can improve accuracy and ability to meet iCGM. In some embodiments, to blank the sensor data, process 700 (e.g., processors) may determine a variable for a CGM calibration algorithm based on the output from the machine learning model and determine whether to blank the sensor based on the CGM calibration algorithm output.

It is contemplated that the steps or descriptions of FIG. 7 may be used with any other embodiment of this disclosure. In addition, the steps and descriptions described in relation to FIG. 7 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-5 could be used to perform one or more of the steps in FIG. 7.

FIG. 8 shows a flowchart of the steps involved in applying machine learning models to detect and correct for erroneous sensor use conditions, in accordance with one or more embodiments. For example, process 800 may represent the steps taken by one or more devices as shown in FIGS. 1-6.

At step 802, process 800 (e.g., using components described in FIGS. 1-6) retrieves a machine learning model that is trained to identify erroneous sensor use conditions based on sensor data error patterns. For example, error patterns may be characteristics (e.g., behaviors, trends, patterns) of the sensor data. For example, in some embodiments, these characteristics of the sensor data may include a downward trend over time (e.g., over the lifetime of a sensor), a spike or drop-off in a signal followed by a return to normal in a short time (e.g., over several seconds), an abnormality that repeats several times over a time period (e.g., several minutes), or other behaviors, trends, or patterns. In some embodiments, the error pattern may depend on a time frame within which it occurs. For example, a certain behavior of a signal may only be characterized as an error pattern if it occurs within a certain time period (e.g., five seconds). In some embodiments, a certain behavior of a signal may only be characterized as an error pattern if it reaches a threshold magnitude (e.g., a minimum required deviation from a normal signal). In some embodiments, a behavior of a signal may only be characterized as an error pattern if it reaches a certain number of repetitions (e.g., three repetitions). Other data characteristics may factor into error pattern detection. In some embodiments, the machine learning model may be trained to recognize error patterns (e.g., such as those described above) using training data. For example, the training data may comprise clinical data on erroneous sensor use conditions. In some embodiments, the training data may additionally include context information (e.g., historic information, sensor data features, etc.) relating to the sensor data. In such cases, sensor data error patterns featuring a known erroneous sensor use condition may be labeled and used to train the system to identify the sensor data error patterns in non-training scenarios. In some embodiments, the system may retrieve a machine learning model that is trained for a particular user based on clinical data from similar users. For example, the system may use clinical data from users of a similar age, height, weight, or level of athleticism, such that the machine learning model is trained to recognized error patterns that are most likely to occur for the user.

At step 804, process 800 (e.g., using components described in FIGS. 1-6) receives CGM sensor data. For example, process 800 may receive the CGM sensor data at a sensor device. For example, the sensor device may be associated with a user device of a user. The sensor data may be real-time sensor data from the sensor device. At step 806, process 800 (e.g., using circuitry described in FIGS. 1-6) inputs the sensor data into the machine learning model.

At step 808, process 800 (e.g., using components described in FIGS. 1-6) receives an output from the machine learning model indicating an erroneous sensor use condition. For example, the erroneous sensor use condition may be based on an error pattern identified in the sensor data. In some embodiments, the erroneous sensor use condition may be further based upon the context information relating to the sensor data. In some embodiments, identified error patterns may be associated with multiple erroneous sensor use conditions.

It is contemplated that the steps or descriptions of FIG. 8 may be used with any other embodiment of this disclosure. In addition, the steps and descriptions described in relation to FIG. 8 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-5 could be used to perform one or more of the steps in FIG. 8.

FIG. 9 shows a machine learning model system for making predictions that facilitate both classification of outlier measurements based on iCGM criteria and detection and correction of erroneous sensor use conditions, in accordance with one or more embodiments.

In some embodiments, the machine learning model system may include one or more neural networks or other machine learning models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it propagates to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free flowing, with connections interacting in a more chaotic and complex fashion.

In some embodiments, the machine learning model system may update its configurations (e.g., weights, biases, or other parameters) based on its assessment of the predictions. Memory may store training data and one or more trained machine learning models.

As an example, a machine learning model 900 may take inputs 902 and provide outputs 904. In one use case, outputs 904 may be fed back (e.g., active feedback) to machine learning model 900 as input to train machine learning model 900 (e.g., alone or in conjunction with user indications of the accuracy of outputs 904, labels associated with the inputs 902, or with other reference feedback information). In another use case, machine learning model 900 may update its configurations (e.g., weights, biases, or other parameters) based on its assessment of its prediction (e.g., outputs 904) and reference feedback information (e.g., user indication of accuracy, reference labels, or other information). In another use case, where machine learning model 900 is a neural network, connection weights may be adjusted to reconcile differences between the neural network's prediction and the reference feedback. In a further use case, one or more neurons (or nodes) of the neural network may require that their respective errors be sent backward through the neural network to them to facilitate the update process (e.g., backpropagation of error). Updates to the connection weights may, for example, be reflective of the magnitude of error propagated backward after a forward pass has been completed. In this way, for example, the machine learning model 900 may be trained to generate better predictions.

For example, in some embodiments, inputs 902 may comprise CGM sensor data (e.g., sensor glucose data), and reference feedback information 904 (which feeds back as input to the machine learning model 900) may include clinical data on iCGM performance. For example, the clinical data may be labeled training data (e.g., labeled as outlier, not outlier). Accordingly, when machine learning model 900 receives a particular glucose measurement as input 902, machine learning model 900 may provide an output 904 including a prediction of whether the measurement is an outlier. This outlier information may be used to correct future signals.

In some embodiments, the system may label the clinical data according to known classifications. In some embodiments, the known classifications may be based upon the FDA iCGM special requirements (e.g., the known classifications may be defined such that the outputs 904 conform to the FDA iCGM special requirements). For example, known classifications may include large negative bias, large positive bias, nominal accuracy, or other labels with respect to the iCGM criteria. For example, a large negative bias may indicate that a particular measurement is underestimated, undervalued, or otherwise too low. A large positive bias may indicate that a particular measurement is overestimated, overvalued, or otherwise too high. In some embodiments, these known classifications may describe the CGM sensor data relative to an average measurement or range across the sensor data, an average measurement or range for sensor data associated with a user of the sensor device, or another reference. In some embodiments, the system may take calibration measurements periodically (e.g., every 4-6 hours or any other predetermined period of time) and derive a reference value from the calibration measurements. The system may thus determine positive and negative bias relative to the reference value. Finally, when machine learning model 900 receives a particular glucose measurement as input 902 to, machine learning model 900 may provide an output 904 including a prediction of whether the measurement corresponds to a large negative bias, large positive bias, or nominal accuracy.

In some embodiments, known classifications may include poor accuracy, intermediate accuracy, good accuracy, or other labels. For example, accuracy labels may correspond to wear times, consistency or inconsistency of readings (e.g., as compared to data for a particular user, training data as a whole, etc.), or other indicators of accuracy. In some embodiments, as described above, the system may take calibration measurements periodically (e.g., every 4-6 hours or any other predetermined period of time) and derive a reference value from the calibration measurements. The system may determine accuracy levels relative to the reference value. Accordingly, when machine learning model 900 receives a particular glucose measurement as input 902 to, machine learning model 900 may provide an output 904 including a prediction of whether the measurement corresponds to a poor accuracy, intermediate accuracy, or good accuracy.

In some embodiments, machine learning model 900 may receive a multi-dimensional feature input based on the sensor data. For example, the inputs 902 received by machine learning model 900 may comprise features such as an EIS feature, Isig trending feature, Isig rate of change feature, previous Isig, previous Vcntr, or other feature inputs. Further description of the above feature inputs can be found in U.S. patent Ser. No. 16/773,422, entitled METHODS, SYSTEMS, AND DEVICES FOR CONTINUOUS GLUCOSE MONITORING, which is herein incorporated by reference in its entirety. In some embodiments, the outputs 904 of machine learning model 900 may depend on combinations of the above feature inputs. For example, certain combinations of Isig, EIS, and Vcntr feature inputs may cause machine learning model 900 to classify the signals as outliers (e.g., or large negative bias, large positive bias, poor accuracy, etc.). In some embodiments, other combinations of Isig, EIS, and Vcntr feature inputs may cause machine learning model 900 to classify the signals as not outliers (e.g., or nominal accuracy, intermediate accuracy, good accuracy, etc.). In some embodiments, the machine learning model may use hit criteria (e.g., as described above) to classify the multi-dimensional feature input as an outlier or not an outlier.

In some embodiments, inputs 902 may comprise sensor data from a sensor device and reference feedback information 904 (which is fed back as input to the machine learning model 900) may include clinical data on erroneous sensor use conditions. For example, the clinical data may be labeled training data, which may include a library of error patterns. In some embodiments, the error patterns may be labeled with corresponding erroneous sensor use conditions. For example, erroneous sensor use conditions may include sensitivity loss, loss of signal, signal spiking, environmental errors (e.g., moisture, heat), or other conditions. Accordingly, when a particular glucose measurement is provided as input 902 to machine learning model 900, machine learning model 900 may provide an output 904 including a prediction of an erroneous sensor use condition based on a detected error pattern.

In some embodiments, inputs 902 may further comprise context information relating to the sensor data. In some embodiments, context information may include pattern definition. For example, error patterns may include combinations of patterns of various sensor input features. In this example, context information may include information about the various components (e.g., sensor input features) of the sensor data and how the error pattern affects these components. In some embodiments, the context information may include historic information relating to the sensor data over a time period. For example, such historic information may include information about behaviors, trends, or patterns of the sensor data over a certain time period leading up to a detected error pattern. In some embodiments, behaviors, trends, or patterns of the sensor data may include downward trends, noisy conditions, a history of error patterns, or other context information. In some embodiments, the time period may be several seconds, minutes, hours, or days, or the time period may be the lifetime of the sensor device. The context information may be important for distinguishing between identical error patterns with different underlying erroneous sensor use conditions. For example, for an error pattern (e.g., low signal) within a first context (e.g., stable history), the system may determine that a first erroneous sensor use condition (e.g., temporary signal loss) is associated with the error pattern. In another example, for the same error pattern (e.g., low signal) within a second context (e.g., downward trend), the system may determine that a second erroneous sensor use condition (e.g., sensitivity loss) is associated with the error pattern. The context information is thus vital to determining the correct erroneous sensor use condition associated with a given error pattern and the correct resolution for correcting the erroneous sensor use condition. In this example, output 904 may be further based upon the context information relating to the sensor data.

In some embodiments, inputs 902 may comprise sensor data from a sensor device and reference feedback information 904 (which is fed back as input to the machine learning model 900) may include data on resolutions associated with various erroneous sensor use conditions. For example, the system may use retrospective techniques to apply resolutions to the training data. The system may determine which resolutions are most effective for resolving each erroneous sensor use condition. Thus, the training data may be labeled training data (e.g., error patterns or erroneous sensor use conditions labeled with effective resolutions). Accordingly, when particular sensor data is provided as input 902 to machine learning model 900, machine learning model 900 may provide an output 904 including a prediction of a resolution that is likely to correct for a detected erroneous sensor use condition.

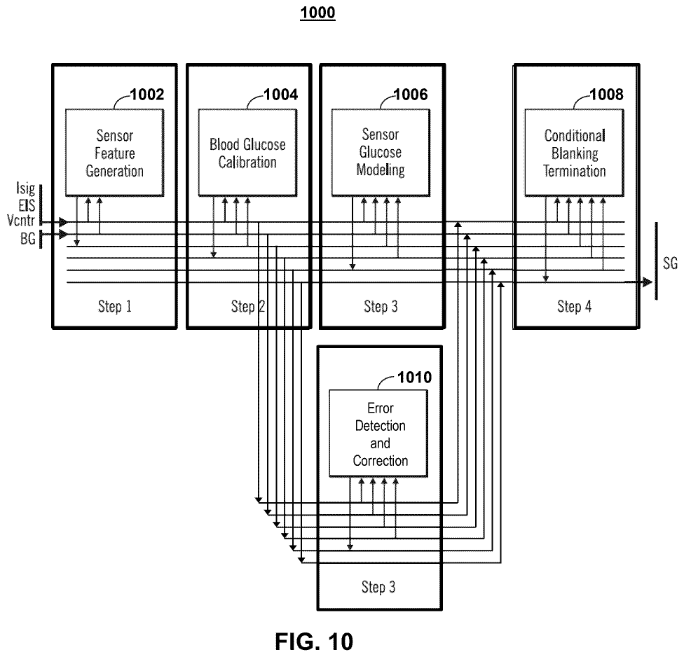

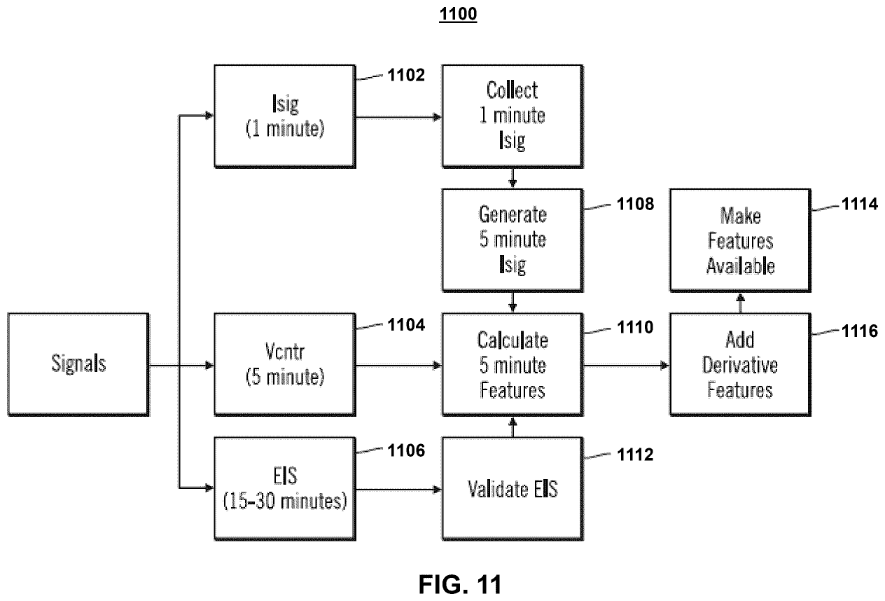

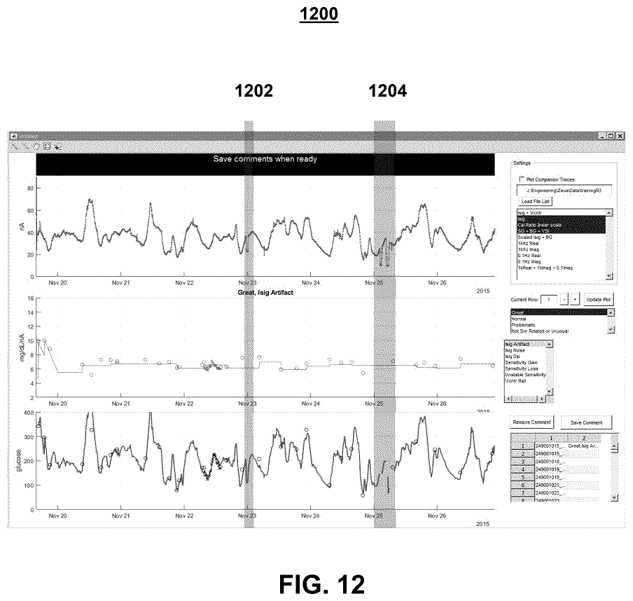

While machine learning model 900 is described in relation to the foregoing examples, it should be understood that the system may train machine learning model 900 to classify or predict characteristics or errors of sensor data according to any other criteria or based on any other inputs. In some embodiments, the system may utilize outputs from machine learning model 900 to determine blanking and termination of signals (e.g., as described below in relation to FIGS. 10 and 11) or to determine resolutions for erroneous sensor use conditions (e.g., as described below in relation to FIGS. 10 and 12).

FIG. 10 shows a flow diagram 1000, in accordance with one or more embodiments. As shown schematically in FIG. 10, the methods and systems described herein include: a sensor feature generator 1002, a blood glucose calibrator 1004, a sensor glucose modeler 1006, a conditional blanker and terminator 1008, and an error detector and corrector 1010. In some embodiments, input data (i.e., interstitially measured current (Isig), counter voltage (Vcntr), electrochemical impedance spectroscopy (EIS), and blood glucose calibration values (BG)) may pass through the algorithm to be transformed to sensor glucose values, or SG. The Table below shows the information input and output from each of the four components.

### Description of the Information Transfer

| Information | Component | Component | Component | Component | Component |
|---|---|---|---|---|---|
| Content | 1002 | 1004 | 1006 | 1008 | 1010 |
| Input signals, Isig, | Input | N/A | N/A | Input | N/A |
| Vcntr, EIS, BG | | | | | |
| Base and | Output | Input | Input | Input | Input |
| Derivative Sensor | | | | | |
| Features Requiring | | | | | |
| No Calibration | | | | | |
| Base and | N/A | Output | Input | Input | Input |
| Derivative Sensor | | | | | |
| Features Requiring | | | | | |
| BG Calibration | | | | | |
| Initial Estimates of | N/A | N/A | Output | Input | Output |
| Sensor Glucose | | | | | |
| Values | | | | | |
| Final Estimates of | N/A | N/A | N/A | Output | N/A |
| Sensor Glucose | | | | | |
| Values | | | | | |

In some embodiments, the input data may flow through components 1002, 1004, 1006, and 1008 (i.e., bypassing component 1010). For example, components 1002, 1004, and 1006 may complete sensor feature generation, BG calibration, and SG modelling, respectively, and component 1008 may classify the sensor data and blank or terminate signals accordingly. In some embodiments, conditional blanker and terminator 1008 may include or be associated with machine learning model 900, as shown in FIG. 9. For example, machine learning model 900 may classify CGM sensor data. For example, machine learning model 900 may classify the sensor data as outlier/not outlier, as having large negative bias/large positive bias/nominal accuracy, or as having poor accuracy/intermediate accuracy/good accuracy. In some embodiments, conditional blanker and terminator 1008 may blank the sensor data (e.g., eschew transmitting sensor data to another device, eschew displaying sensor data on a display interface) based on the output from machine learning model 900. For example, if the output from machine learning model 900 indicates that a measurement from the sensor data is not an outlier (e.g., does not breach a threshold), conditional blanker and terminator 1008 may not blank that measurement. Similarly, if the output from machine learning model 900 indicates that a measurement has intermediate, normal, or good accuracy, conditional blanker and terminator 1008 may not blank that particular measurement. In some embodiments, if the output from machine learning model 900 indicates that a measurement from the sensor data is an outlier, conditional blanker and terminator 1008 may blank that measurement. Similarly, if the output from machine learning model 900 indicates that a measurement has a large bias (e.g., positive or negative) or has poor accuracy, conditional blanker and terminator 1008 may blank that measurement. In some embodiments, conditional blanker and terminator 1008 may blank certain measurements based on any other classifications output by machine learning model 900.

In some embodiments, blanking the CGM sensor data (e.g., via a display interface) may include determining a variable for the CGM calibration algorithm based on the output from machine learning model 900 (e.g., as shown in FIG. 7). In some embodiments, the variable for the CGM calibration algorithm may depend on whether machine learning model 900 outputs an outlier classification (e.g., nominal accuracy, intermediate accuracy, good accuracy, etc.) or a non-outlier classification (e.g., large positive bias, large negative bias, poor accuracy, etc.). In some embodiments, conditional blanker and terminator 1008 may determine whether to blank the sensor data based on the output from the CGM calibration algorithm.

In some embodiments, conditional blanker and terminator 1008 may additionally or alternatively terminate a sensor (e.g., stop transmitting any sensor data from that sensor and/or stop displaying any sensor data from that sensor). For example, conditional blanker and terminator 1008 may monitor measurements that have been blanked (i.e., outliers). Conditional blanker and terminator 1008 may track a number of consecutive blanked measurements (e.g., within a certain time frame). For example, conditional blanker and terminator 1008 may use an outlier counter to track blanked measurements and determine when to terminate a sensor. In some embodiments, when machine learning model 900 classifies a measurement as not an outlier, the system may reset the outlier counter (e.g., to zero). In some embodiments, when machine learning model 900 classifies a certain number of measurements in a row as not outliers, the system may reset the outlier counter. For example, machine learning model 900 classifies five measurements as not outliers, the system may reset the outlier counter. In some embodiments, the system may reset the outlier counter based on measurements that machine learning model 900 classifies as having nominal accuracy, intermediate accuracy, good accuracy, etc.

If conditional blanker and terminator 1008 identifies a measurement that has been classified as an outlier (e.g., or large positive bias, large negative bias, poor accuracy, etc.) by machine learning model 900, the system may increase the outlier counter. For example, the system may increment the outlier counter by one. In this example, the outlier counter may continue to increase when outlier measurements are received. As described above, the system may reset the outlier counter at any time in response to receiving one or more measurements classified as non-outliers. The outlier counter may continue to increase in response to outlier measurements until a threshold is breached. For example, the threshold may be predetermined. In some embodiments, the threshold may dynamically change based on wear time, battery life, or other factors. In some embodiments, the threshold may have an associated time limit. For example, in some embodiments, the outlier counter must breach the threshold within a certain time frame (e.g., 1 hour) in order to terminate the signal (e.g., stop transmitting any sensor data from that sensor and/or stop displaying any sensor data from that sensor). In some embodiments, once the system terminates the signal, conditional blanker and terminator 1008 may alert the user that the sensor must be replaced (e.g., via a display interface).

FIG. 11 shows the schematic 1100 of the sensor feature generator 1002 of FIG. 10, in accordance with one or more embodiments. As shown, the consumes the following input signals: Isig (e.g., 1-minute) 1102, Vcntr (e.g., 5-minute) 1104, and EIS (e.g., 15-30 minutes) 1106. The Isig signals are collected over 5 minutes to generate a 5-minute Isig signal 1108 and the EIS signal is validated at 1112. The latter two signals are then used, along with preprocessing, to generate 5-minute and derivative features (e.g., 1110, 1116). In some embodiments, measurement frequency of signals can change depending on the hardware design, e.g., Vcntr may be measured at 1-minute and EIS may be measured more frequently, depending on battery life and memory size limitations. The Table below provides a description of the input signals to sensor feature generator 1002. Sensor feature generator 1002 will make these input signals, as well as the signals it generates, available to blood glucose calibrator 1004, sensor glucose modeler 1006, and conditional blanker and terminator 1008 (e.g., at 1114).

### Description of the Input Signals to Sensor Feature Generator 1002

| | | | | Output |
|---|---|---|---|---|
| | | Input Time | | Time |
| Signal | Description | Lapse | | Lapse |
| Isig | Interstitial Current Signal | 1 | minute | 5 minutes |
| Vcntr | Counter Voltage Signal | 5 | minutes | 5 minutes |
| EIS | Electrochemical Impedance | 15 to 30 | minutes | 5 minutes |
| | Spectroscopy Signal | | | |

As shown in FIG. 11, the system may process the Isig, Vcntr, and EIS signals based on the first time interval (e.g., 5-minute features 1110). In some embodiments, the packet, including all or some of these signals, may be used to generate a calibrated SG value, which is displayed to the user (e.g., via a display interface). At any point in each interval, the system may blank the output values provided to the user (e.g., as described above in relation to FIG. 10) in response to the system classifying a signal included in the interval as an outlier. In some embodiments, the outlier counter described above may utilize the first interval to reset or increase. In other words, the system may reset the outlier counter or increase the outlier counter based identifying a measurement included in the interval as an outlier. An aggregation of packets including blanked signals (e.g., multiple consecutive five-minute increments) may cause the system to terminate the sensor (e.g., stop transmitting any sensor data from that sensor and/or stop displaying any sensor data from that sensor).

Returning to FIG. 10, error detector and corrector 1010 may function in parallel with sensor glucose modeler 1006. For example, error detector and corrector 1010 may take the same inputs as sensor glucose modeler 1006 (e.g., as shown in the Table above). In some embodiments, outputs from error detector and corrector 1010 may, at times, feed into conditional blanker and terminator 1008. For example, if error detector and corrector 1010 detects no error patterns in the sensor data, the output from sensor glucose modeler 1006 may function as the initial estimates of sensor glucose values, as input into conditional blanker and terminator 1008. If error detector and corrector 1010 detects an error pattern in the sensor data, the system may switch over such that the output from error detector and corrector 1010 functions as the initial estimates of sensor glucose values, as input into conditional blanker and terminator 1008. Thus, while sensor glucose modeler 1006 and error detector and corrector 1010 may process the sensor data in parallel, the output from sensor glucose modeler 1006 may be the primary input into conditional blanker and terminator 1008 under circumstances in which no error patterns are detected in the sensor data and the output from error detector and corrector 1010 may be the primary input into conditional blanker and terminator 1008 under circumstances in which one or more error patterns are detected in the sensor data. In other words, if error detector and corrector 1010 detects no error patterns in the signal, flow diagram 1000 may function as if it were independent of error detector and corrector 1010.

In some embodiments, error detector and corrector 1010 may identify a resolution in response to a detected erroneous sensor use condition. For example, error detector and corrector 1010 may identify a resolution that is associated with the detected erroneous sensor use condition. In some embodiments, the resolution may be associated with the detected erroneous sensor use condition via a database, library of patterns, library of resolutions, or by some other means. In some embodiments, the resolution may be associated with the detected erroneous sensor use condition via an output from a machine learning model (e.g., machine learning model 900), as discussed above in relation to FIG. 9. In some embodiments, error detector and corrector 1010 may implement the identified resolution or may cause the identified resolution to be implemented (e.g., by another component). In some embodiments, the system may implement the identified resolution by manipulating the sensor glucose data that is displayed on a user device (e.g., via a display interface). For example, the system (e.g., conditional blanker and terminator 1008) may blank the sensor glucose data from the sensor device. In another example, the system may flag a user of the sensor device (e.g., by generating an alert, informing the user of the erroneous sensor use condition, requesting an action from a user, etc.). In some embodiments, the system (e.g., error detector and corrector 1010) may implement the identified resolution by manipulating (e.g., processing) the sensor data directly. For example, error detector and corrector 1010 may adjust a signal, add a filter to a signal, adjust a filter of a signal, replace a signal (e.g., with a trending signal, a signal from a library, etc.), or otherwise manipulate or process the sensor data.

In some embodiments, error detector and corrector 1010 may include or be associated with machine learning model 900, as shown in FIG. 9. For example, machine learning model 900 may detect error patterns in the sensor data or identify a resolution in response to a detected erroneous sensor use condition. In some embodiments, machine learning model 900 may generate additional predictions which error detector and corrector 1010 may utilize for identifying and correcting erroneous sensor use conditions.

Note that, in some embodiments, a measurement that is determined to be an outlier may be blanked, such that the measurement is not displayed and/or is not used by a medical device (e.g., an insulin infusion pump) to determine medication (e.g., insulin) dosages. In some instances in which measurements from a sensor are blanked, the sensor may be able to recover. However, there may be instances where a sensor cannot recover and/or is determined to be damaged or malfunctioning in a way that measurements cannot be used. Such a sensor may be terminated. In response to determining that a sensor is to be terminated, the sensor may inhibit transmission of measurements to paired devices (e.g., a paired mobile phone or controller device, a paired insulin infusion pump, etc.) and/or may cause an alert to be presented instructing a user to stop using the terminated sensor.

In some embodiments, an outlier counter may be incremented in response to determining that a measurement is an outlier, and, in some cases, is to be blanked. Continuing with this example, the outlier counter may be used to determine that a sensor is to be terminated, e.g., if a count of the outlier counter exceeds a predetermined threshold number and/or exceeds a predetermined threshold number over a predetermined time frame (e.g., more than X outlier measurements, more than Y outlier measurements in Z minutes, etc.). Conversely, an outlier counter may be reset (e.g., to zero) responsive to determining that an updated measurement (e.g., a measurement made after a previous measurement determined to be an outlier) is not an outlier. Accordingly, the techniques disclosed herein may utilize an outlier counter to monitor, over time, sensor measurements to assess whether a sensor has “recovered” after generating one or more outlier measurements, or, conversely, whether the sensor is to be terminated. Note that use of the outlier counter may enable extended use of a glucose sensor by allowing assessment of whether a sensor that has generated outlier measurements has recovered, and therefore, need not be terminated. This in turn extends patient comfort (by not requiring patients to unnecessarily remove a terminated sensor and insert a new sensor), reduces waste of sensors that have recovered after generating one or more outlier measurements, etc.

Note that, in some embodiments, a trained machine learning model may be utilized to generate an output indicating whether a sensor is to be terminated. Such a machine learning model may have a similar structure to a model trained to determine whether a sensor measurement is an outlier, but may be trained based on different training data to generate a different output (e.g., whether the sensor is to be terminated). In some implementations, a model that is trained to generate an output indicating whether a sensor is to be terminated may take, as input, a multi-dimensional feature vector. The multi-dimensional feature vector may include electrical parameters (e.g., iSig values, Vctr values, EIS values, etc.), as shown in and described above in connection with FIG. 11. Additionally or alternatively, in some implementations, the multi-dimensional feature vector may include manufacturing information associated with the sensor, such as a make/model of the sensor, a lot of the sensor, an age of the sensor, information regarding chemical composition or other information regarding the physical structure of the sensor, calibration information measured on a test sensor of the same lot or manufactured from the same substrate (e.g., in vitro iSIG, Vctr, and/or EIS data), or the like. A training set used to train such a model may include training samples that each include a multi-dimensional feature vector that includes data from a sensor, and a ground truth indication of whether the sensor is to be terminated or not. Accordingly, the model may be trained to classify a given sensor, at inference time, as to be terminated, or not to be terminated based on multi-dimensional feature vector data.

FIG. 12 shows a graph 1200 highlighting error patterns in sensor data, in accordance with one or more embodiments. As shown in graph 1200, the system (e.g., error detector and corrector 1010, as shown in FIG. 10) may identify various error patterns, such as error pattern 1202 and error pattern 1204. Both error pattern 1202 and error pattern 1204 may demonstrate a dropping off of a signal. In this example, error detector and corrector 1010 may identify an underlying erroneous sensor use condition for each error pattern based on the error pattern, context information of the sensor data (e.g., as described above), or other information relating to the sensor data.

In some embodiments, a hybrid system may be used which determines whether an outlier measurement from a sensor can be compensated rather than being blanked. If a determination is made that the measurement may be compensated, such compensation may be performed to generate a compensated measurement, where the compensated measurement is not blanked and is utilized as normal (e.g., displayed on a display device, transmitted to a medical device to determined medication dosage, etc.). By way of example, it may be determined that a sensor has generated one or more outlier measurements (e.g., using the techniques shown in and described above in connection with FIGS. 7-11), e.g., due to sensitivity loss, sensor underread or sensor overread, etc. Continuing with this example, it may be determined that the particular type of sensor malfunctioning (e.g., sensitivity loss, sensor underread, sensor overread, etc.) and/or a degree of malfunctioning can be safely compensated for. Accordingly, a compensation may be performed on the sensor measurement rather than blanking the measurement. In some embodiments, the sensor may continue to be monitored to be determined whether the sensor is to be terminated. For example, a determination may be made that the degree of compensation has increased over time, and accordingly, the sensor should be terminated. Note that, in some embodiments, a determination of whether particular outlier measurements can be compensated, and the compensation itself, may be determined using one or more trained machine learning models. Similar to what is shown in and described above in connection with FIG. 11, the machine learning model(s) may take, as input, a multi-dimensional feature vector that includes in vivo and/or in vitro information, which may include in vivo electrical parameters (e.g., iSig, Vctr, and/or EIS measurements), manufacturing information, and/or in vitro calibration measurements (e.g., iSig, Vctr, and/or EIS measurements made in vitro using the sensor as part of the manufacturing process, and/or made using a test sensor from the same lot or from the same substrate).

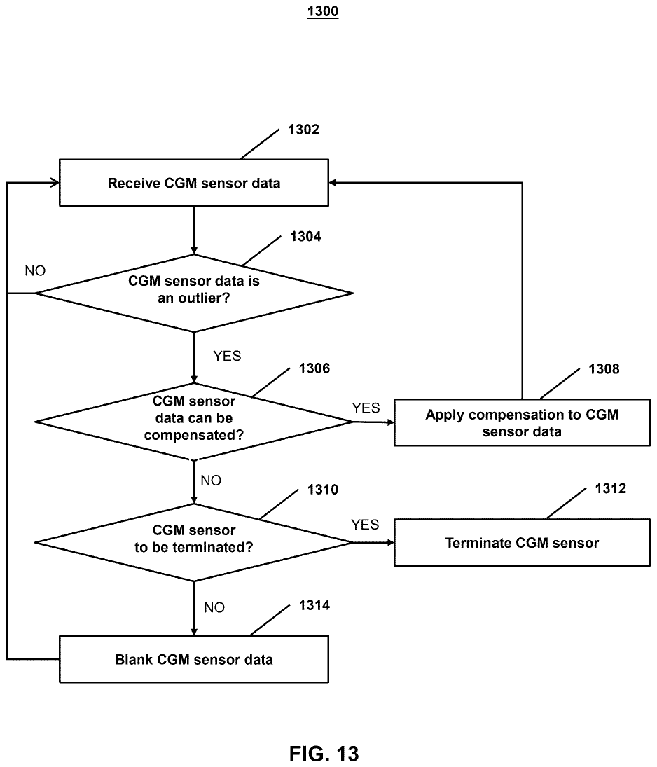

FIG. 13 shows a flowchart of an example process for compensating outlier sensor measurements in accordance with one or more embodiments. Blocks of process 1300 may be performed by one or more processors, such as one or more processors of the devices shown in and described above in connection with FIGS. 1-6.

Process 1300 can begin at 1302 by receiving CGM sensor data. The sensor data may be acquired from a CGM sensor that is at least partially subcutaneously inserted in a patient. In other words, the received CGM sensor data may be in vivo sensor data. The CGM sensor data may include iSIG values, Vctr values, EIS values, etc.

At 1304, process 1300 can determine whether the CGM sensor data includes an outlier measurement. For example, as shown in and described above in connection with FIGS. 7-11, process 1300 may provide a representation of the received CGM sensor data to a trained machine learning model trained to generate an output classifying the CGM sensor data as including an outlier measurement, or not. The representation of the received CGM sensor data may include multiple features, which may be aggregated into a multi-dimensional feature vector. The multiple features may include in vivo data (e.g., in vivo iSIG, Vctr, and/or EIS data), manufacturing information associated with the sensor, and/or in vitro data (e.g., obtained during a calibration process associated with manufacturing from the CGM sensor, and/or from a CGM sensor that is from the same lot and/or manufactured from the same substrate).

In some embodiments, a machine learning model that determines whether a sensor measurement is an outlier may be trained using a training data set that includes pairs of blood glucose (e.g., measured using a fingerstick measurement) and corresponding CGM sensor measurements, in conjunction with, for each pair, a label that indicates the pair as an outlier or as not an outlier. Accordingly, the machine learning model may be trained to predict the training label in order to label CGM sensor data as an outlier or not an outlier. By way of example, a training set may include blood glucose measurements of [50, 100, 150] and corresponding CGM sensor measurements of [52, 101, 200]. The pairs of (50, 52) and (100, 101) may be labeled in the training data as “not outlier,” while the pair of (150, 200) may be labeled as “outlier,” because the difference between 150 and 200 in the pair of (150, 200) exceeds a threshold. Note that, the model may be trained using blood glucose data (e.g., fingerstick data) to allow the model to learn, during training, characteristics or features of CGM sensor data that may be indicative of an outlier. Once the model has been trained (e.g., using paired blood glucose data and CGM sensor data), the model need not utilize blood glucose data (e.g., fingerstick data) at inference time. By way of example, during training, the model may be trained to learn that particular characteristic combinations CGM sensor data features (e.g., a combination of Isig and Vctr values, a combination of Isig and EIS feature values, etc.) tend to result in an outlier, where, during training, the pairing of the blood glucose value with the CGM sensor data allow the model to be trained to identify outlier measurements, and, more particularly, characteristics of CGM sensor data indicative of an outlier. At inference time, the learned characteristics of the CGM sensor data may be used to identify outliers in the CGM sensor data without corresponding blood glucose data.

If, at 1304, process 1300 determines that the CGM sensor data does not include an outlier measurement (“no” at 1304), process 1300 can loop back to block 1302 and can receive additional CGM sensor data.

Conversely, if, at 1304, process 1300 determines that the CGM sensor data does include an outlier measurement (“yes” at 1304), process 1300 can proceed to block 1306 and can determine whether the CGM sensor data can be compensated. In some embodiments, process 1300 can determine whether the CGM sensor data can be compensated by providing a representation of the received CGM sensor data to a trained machine learning model. Similar to what is described above in connection with block 1304, the representation of the received CGM sensor data may include multiple features, which may be aggregated into a multi-dimensional feature vector. The multiple features may include in vivo data (e.g., in vivo iSIG, Vctr, and/or EIS data), manufacturing information associated with the sensor, and/or in vitro data (e.g., obtained during a calibration process associated with manufacturing from the CGM sensor, and/or from a CGM sensor that is from the same lot and/or manufactured from the same substrate). Note that, in some embodiments, the trained machine learning model that determines whether the CGM sensor data can be compensated may be different from the model that determines whether the CGM sensor data includes an outlier measurement. In some embodiments, the model that determines whether sensor data can be compensated may be different from a model that determines whether the sensor data includes outlier measurements, however, in some such embodiments, the input to each model may be at least partially the same. For example, the same multi-dimensional feature vector may be provided as input to both models.

In some implementations, a training set used to train a model to determine whether sensor data can be compensated may be similar to, the same as, or a subset of the training data used to train a model to determine whether sensor data is an outlier. However, the training data may be labeled differently, e.g., to indicate whether or not particular data can be compensated for or not. Similar to what is described above, a model that is trained to determine whether particular data may be compensated may be trained using a training set that includes pairs of CGM sensor data and corresponding blood glucose data (e.g., fingerstick data), as well as training labels indicating whether the CGM sensor data can be compensated and/or a manner in which compensation may occur. For example, the blood glucose data may allow the model to learn a degree of difference between a blood glucose measurement and a corresponding CGM sensor data that can be compensated and relate the degree of difference to characteristics of the CGM sensor data. As discussed above, characteristics of the CGM sensor data may include multiple features of the CGM sensor data (e.g., Isig, Vctr, EIS measurements, etc.). At inference time, the learned characteristics of the CGM sensor data may be used by the trained machine learning model to identify outlier CGM sensor data that may be compensated.

Note that a model that determines whether CGM sensor data can be compensated may be trained to identify particular types of errors or error patterns (e.g., a loss of sensitivity, sensor underread, sensor overread, etc.) and may be trained to determine that certain of those error types may be compensated for, while other error types may not be compensated for. In some embodiments, the model may be trained to determine whether outliers within a particular range can be compensated for. Note that, in some embodiments, a model that determines whether CGM sensor data may be compensated for may be trained using iCGM criteria, thereby ensuring that any compensation applied adheres to iCGM safety criteria.

If, at 1306, process 1300 determines that the CGM sensor data can be compensated (“yes” at 1306), process 1300 can proceed to block 1308 and can apply compensation to the CGM sensor data. Applying compensation may be dependent on the type of outlier. For example, if the outlier measurement is determined to correspond to sensitivity loss, where CGM sensor data is overly noisy, compensation may involve filtering the CGM sensor data. As another example, if the outlier measurement is determined to correspond to a sensor underread, compensation may involve scaling the CGM sensor data by a factor (e.g., a factor greater than 1). As yet another example, if an outlier measurement is determined to correspond to a sensor overread, compensation may involve scaling the CGM sensor data by a factor (e.g., a factor less than 1). Note that the type of compensation to be applied and a magnitude of the compensation may be determined based on an output of the trained machine learning model that determines that compensation may be performed.

In some embodiments, determining compensation for an outlier measurement may involve balancing compensating outliers to a nominal range while avoiding compensating false positive outliers. For example, if an outlier corresponds to a sensor overread, and the overread is estimated as, e.g., 50 mg/dL, a compensation of −50 mg/dL may bring the outlier measurement back to a nominal range. However, if the detected outlier is in reality a false positive outlier (e.g., a measurement detected as an outlier that is not truly an outlier), applying such a large compensation will create compensated data that is inaccurate. In some embodiments, a hyperparameter tuning technique or other optimization process may be used to identify compensation parameters that compensate outlier measurements to bring them to a nominal range without hurting overall performance (e.g., by compensating false positive outliers). In one example, the hyperparameter tuning technique is a grid search. Using a grid search technique, a hyperparameter space may be defined that includes different compensation parameters, and a grid may be formed that includes all possible values of the compensation parameters. A model may be trained for each combination in the grid using training data, and the best-performing model may be selected. Note that the best-performing model may be identified as one that most compensates outlier measurements to a nominal range while avoiding compensation for false positive outliers. In such cases, the training data may include true outliers and false positive outliers to allow for identification of the best-performing model. Alternative techniques to a grid search include a random search, Bayesian optimization, successive halving or early stopping, genetic algorithms, or the like.

After applying compensation to the CGM sensor data, process 1300 can loop back to block 1302 and can receive additional CGM sensor data. Note that the compensated sensor data is not blanked, and accordingly, may be used as normal (e.g., may be displayed, used to calculate insulin dosages, etc.).

If, at 1306, process 1300 determines that the CGM sensor data cannot be compensated (“no” at 1306), process 1300 can determine, at 1310, whether the CGM sensor is to be terminated. Determining whether the CGM sensor is to be terminated may be based on an outlier counter, provision of a representation of CGM sensor data to a trained machine learning model trained to generate an output indicative of whether the CGM sensor is to be terminated, or any combination thereof. Note that, in some embodiments, process 1300 may consider a degree to which the CGM sensor data has been compensated and/or a magnitude of sensor drift in the determination of whether to terminate. For example, in an instance in which process 1300 determines that the CGM sensor data has been compensated by increasing degree (e.g., scaled by increasingly larger factors to compensate for sensor underread, scaled by increasingly smaller factors to compensate for sensor overread, etc.), process 1300 may determine that the sensor is to be terminated. As a more particular example, process 1300 may determine a rate of change of compensation, and may determine the sensor is to be terminated if the rate of change of compensation exceeds a predetermined threshold.

Note that, determinations of whether sensor data should be blanked, terminated, and/or compensated may each be implemented as different modules, each associated with a different trained machine learning model that analyzes the sensor data. The modules may operate concurrently such that as data packets that include sensor data are received, each packet is analyzed, sometimes by all three modules. In some embodiments, modules may communicate with one another. For example, a module that determines whether a sensor is to be terminated may receive information from a module that indicates how many times data from the sensor has been blanked (e.g., received from a blanking module) and/or data that indicates how many times and/or to what degree sensor data has been compensated (e.g., data received from a compensation module). Accordingly, a decision to terminate a sensor may consider sensor data, historical or previous decisions regarding sensor blanking, and/or historical or previous decisions regarding sensor data compensation.

If, at 1310, process 1300 determines that the CGM sensor is to be terminated (“yes” at 1310, process 1300 can terminate the sensor. For example, process 1300 may inhibit transmission of CGM sensor data to other paired devices (e.g., a paired controller device such as a mobile phone and/or insulin pump). As another example, process 1300 may cause an alert to be presented requesting that a user of the CGM sensor change the CGM sensor.

Conversely, if, at 1310, process 1300 determines that the CGM sensor is not to be terminated (“no” at 1310), process 1300 can blank the CGM sensor data at 1314. For example, process 1300 can transmit the CGM sensor data to other paired devices (e.g., a paired controller device such as a mobile phone and/or insulin pump) concurrent with an instruction to not display the data and/or not use the sensor data to calculate insulin dosages. Process 1300 can then loop back to block 1302 and can receive additional CGM sensor data.

Process 1300 can loop through blocks 1302-1314 until either the CGM sensor is terminated (e.g., at block 1312), or until a normal lifespan of the CGM sensor has elapsed. Once the normal lifespan of the CGM sensor has elapsed, a user of the CGM sensor data may be instructed to change the CGM sensor.

The above-described embodiments of the present disclosure are presented for purposes of illustration and not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

The present techniques will be better understood with reference to the following enumerated embodiments:

1. A method comprising: receiving, at a sensor device, CGM sensor data; inputting, at the sensor device, the sensor data in a machine learning model, wherein the machine learning model is trained to identify outlier measurements based on sensor glucose-dependent performance against iCGM criteria using training data comprising clinical data on iCGM performance; receiving, at the sensor device, an output from the machine learning model indicating that the sensor data corresponds to an outlier measurement of the outlier measurements; and blanking the sensor data based on the output.

2. The method of embodiment 1, wherein the machine learning model is trained using a set of training sensor data that is labeled according to known classifications, the known classifications comprising a large negative bias, large positive bias, or nominal accuracy.

3. The method of any of embodiments 1-2, wherein the machine learning model is trained using a set of training sensor data that is labeled according to known classifications, the known classifications comprising poor accuracy, intermediate accuracy, or good accuracy.

4. The method of any of embodiments 1-3, wherein the sensor data comprises a current signal, a voltage signal, or impedance spectroscopy signals.

5 The method of embodiment 4, wherein inputting the sensor data in the machine learning model comprises generating a multi-dimensional feature input based on the sensor data.

6. The method of any of embodiments 1-5, wherein blanking the sensor data based on the output further comprises: determining a variable for a CGM calibration algorithm based on the output; and determining whether to blank the sensor data based on the output from the from the machine learning model.

7. The method of any of embodiments 1-6, wherein the sensor data is received in first time intervals.

8. The method of any of embodiments 1-7, further comprising: resetting an outlier counter based on determining that a first sensor datapoint does not correspond to an outlier measurement; causing the outlier counter to be increased based on determining that a second sensor datapoint corresponds to an outlier measurement; comparing the outlier counter to a threshold; and terminating the sensor device based on determining that the outlier counter has breached the threshold.

9. A method comprising: receiving, at a sensor device, CGM sensor data; inputting, at the sensor device the sensor data into a machine learning model, wherein the machine learning model is trained to identify erroneous sensor use conditions based sensor data error patterns using training data comprising clinical data on erroneous sensor use conditions; and receiving, at the sensor device, an output from the machine learning model indicating an erroneous sensor use condition based on an error pattern identified in the sensor data.

10. The method of embodiment 9, further comprising blanking the sensor data based on the output.

11. The method of any of embodiments 9-10, further comprising flagging a user of the sensor device, on a display interface, based on the output.

12. The method of any of embodiments 9-11, further comprising identifying a resolution associated with the erroneous sensor use condition identified in the sensor data.

13. The method of embodiment 12, further comprising implementing the resolution by manipulating the sensor data, on a display interface, of the sensor device.

14. The method of embodiment 12, further comprising implementing the resolution by manipulating the sensor data received from the sensor device.

15. The method of any of embodiments 9-14, further comprising: receiving input indicating context information relating to the sensor data; inputting the context information relating to the sensor data into the machine learning model; and wherein the output from the machine learning model indicating an erroneous sensor use condition is further based on the context information relating to the sensor data.

16. The method of embodiment 15, wherein the context information relating to the sensor data includes historic information relating to the sensor data over a time period.

17. The method of embodiment 9, further comprising training the machine learning model to identify the erroneous sensor use conditions based on the sensor data error patterns using the training data comprising the clinical data on the erroneous sensor use conditions.

18. A tangible, non-transitory, machine-readable medium storing instructions that, when executed by a data processing apparatus, cause the data processing apparatus to perform operations comprising those of any of embodiments 1-17.

19. A system comprising: one or more processors; and memory storing instructions that, when executed by the processors, cause the processors to effectuate operations comprising those of any of embodiments 1-17.

20. A system comprising means for performing any of embodiments 1-17.

21. A system comprising: one or more processors; and one or more processor-readable media storing instructions which, when executed by the one or more processors, cause performance of: obtaining sensor data; applying, to the sensor data, a machine learning model trained to identify outlier measurements; determining, based on output from the machine learning model, that the sensor data corresponds to an outlier measurement; and blanking the sensor data based on the output.

22. The system of embodiment 21, wherein the output comprises a known classification from a set of three or more known classifications including a large negative bias classification, a large positive bias classification, and a nominal accuracy classification.

23. The system of any one of embodiments 21 or 22, wherein the output comprises a known classification from a set of three or more known classifications including a poor accuracy classification, an intermediate accuracy classification, and a good accuracy classification.

24. The system of any one of embodiments 21-23, wherein the sensor data comprises a combination of in vivo sensor data obtained while a sensor from which the sensor data is in use by a patient and in vitro data obtained from the sensor or a sensor manufactured from a same manufacturing batch as the sensor.

25. The system of any one of embodiments 21-24, wherein applying the machine learning model comprises generating a multi-dimensional feature input based on the sensor data.

26. The system of any one of embodiments 21-25, wherein blanking the sensor data comprises: transmitting the sensor data to a paired device in connection with an indication that the sensor data is not to be used for determination of insulin dosages.

27. The system of any one of embodiments 21-26, wherein blanking the sensor data comprises eschewing transmission of the sensor data to a paired device.

28. The system of any one of embodiments 21-27, wherein the one or more processor-readable media further store instructions which, when executed by the one or more processors, cause performance of: resetting an outlier counter based on determining that a first sensor datapoint does not correspond to an outlier measurement; increasing the outlier counter based on determining that a second sensor datapoint corresponds to an outlier measurement; comparing the outlier counter to a threshold; and terminating a sensor device based on determining that the outlier counter has breached the threshold.

29. The system of any one of embodiments 21-28, wherein the machine learning model was trained based on integrated continuous glucose monitoring (iCGM) criteria.

30. A processor-implemented method comprising: obtaining sensor data; applying, to the sensor data, a machine learning model trained to identify outlier measurements; determining, based on output from the machine learning model, that the sensor data corresponds to an outlier measurement; and blanking the sensor data based on the output.

31. The processor-implemented method of embodiment 30, wherein the output comprises a known classification from a set of three or more known classifications including a large negative bias classification, a large positive bias classification, and a nominal accuracy classification.

32. The processor-implemented method of any one of embodiments 30 or 31, wherein the output comprises a known classification from a set of three or more known classifications including a poor accuracy classification, an intermediate accuracy classification, and a good accuracy classification.

33. The processor-implemented method of any one of embodiments 30-32, wherein the sensor data comprises a combination of in vivo sensor data obtained while a sensor from which the sensor data is in use by a patient and in vitro data obtained from the sensor or a sensor manufactured from a same manufacturing batch as the sensor.

34. The processor-implemented method of any one of embodiments 30-33, wherein applying the machine learning model comprises generating a multi-dimensional feature input based on the sensor data.

35. The processor-implemented method of any one of embodiments 30-34, wherein blanking the sensor data comprises transmitting the sensor data to a paired device in connection with an indication that the sensor data is not to be used for determination of insulin dosages.

36. The processor-implemented method of any one of embodiments 30-35, further comprising: updating an outlier counter responsive to determining that the sensor data corresponds to an outlier measurement; comparing a value of the outlier counter to a threshold; and in response to determining that the value of the outlier counter exceeds the threshold, terminating a sensor device from which the sensor data was obtained.

37. The processor-implemented method of any one of embodiments 30-36, further comprising: after determining that the sensor data corresponds to the outlier measurement, determining whether the sensor data can be compensated; in response to determining that the sensor data cannot be compensated, blanking the sensor data.

38. A processor-implemented method comprising: obtaining sensor data; applying, to the sensor data, a first machine learning model trained to identify outlier measurements; determining, based on output from the machine learning model, that the sensor data corresponds to an outlier measurement; in response to determining that the sensor data corresponds to the outlier measurement, applying, to the sensor data, a second machine learning model trained to identify whether the outlier measurement can be compensated; determining, based on output from the second machine learning model, that the outlier measurement can be compensated; and in response to determining that the outlier measurement can be compensated, applying a compensation to the sensor data to generate compensated sensor data.

39. The processor-implemented method of embodiment 38, wherein applying the compensation to the sensor data is instead of blanking the sensor data.

40. The processor-implemented method of any one of embodiments 38 or 39, wherein the second machine learning model is configured to take, as input, a combination of in vivo sensor data obtained while a sensor from which the sensor data is in use by a patient and in vivo data obtained from the sensor or a sensor manufactured from a same substrate as the sensor.

What is claimed is:

1. A system comprising:
one or more processors; and
one or more processor-readable media storing instructions which, when executed by the one or more processors, cause performance of:
obtaining sensor data;
applying, to the sensor data, a machine learning model trained to identify outlier measurements;
determining, based on output from the machine learning model, that the sensor data corresponds to an outlier measurement; and
blanking the sensor data based on the output.

2. The system of claim 1, wherein the output comprises a known classification from a set of three or more known classifications including a large negative bias classification, a large positive bias classification, and a nominal accuracy classification.

3. The system of claim 1, wherein the output comprises a known classification from a set of three or more known classifications including a poor accuracy classification, an intermediate accuracy classification, and a good accuracy classification.

4. The system of claim 1, wherein the sensor data comprises a combination of in vivo sensor data obtained while a sensor from which the sensor data is in use by a patient and in vitro data obtained from the sensor or a sensor manufactured from a same manufacturing batch as the sensor.

5. The system of claim 1, wherein applying the machine learning model comprises generating a multi-dimensional feature input based on the sensor data.

6. The system of claim 1, wherein blanking the sensor data comprises:
transmitting the sensor data to a paired device in connection with an indication that the sensor data is not to be used for determination of insulin dosages.

7. The system of claim 1, wherein blanking the sensor data comprises eschewing transmission of the sensor data to a paired device.

8. The system of claim 1, wherein the one or more processor-readable media further store instructions which, when executed by the one or more processors, cause performance of:
resetting an outlier counter based on determining that a first sensor datapoint does not correspond to an outlier measurement;
increasing the outlier counter based on determining that a second sensor datapoint corresponds to an outlier measurement;
comparing the outlier counter to a threshold; and
terminating a sensor device based on determining that the outlier counter has breached the threshold.

9. The system of claim 1, wherein the machine learning model was trained based on integrated continuous glucose monitoring (iCGM) criteria.

10. A processor-implemented method comprising:
obtaining sensor data;
applying, to the sensor data, a machine learning model trained to identify outlier measurements;
determining, based on output from the machine learning model, that the sensor data corresponds to an outlier measurement; and
blanking the sensor data based on the output.

11. The processor-implemented method of claim 10, wherein the output comprises a known classification from a set of three or more known classifications including a large negative bias classification, a large positive bias classification, and a nominal accuracy classification.

12. The processor-implemented method of claim 10, wherein the output comprises a known classification from a set of three or more known classifications including a poor accuracy classification, an intermediate accuracy classification, and a good accuracy classification.

13. The processor-implemented method of claim 10, wherein the sensor data comprises a combination of in vivo sensor data obtained while a sensor from which the sensor data is in use by a patient and in vitro data obtained from the sensor or a sensor manufactured from a same manufacturing batch as the sensor.

14. The processor-implemented method of claim 10, wherein applying the machine learning model comprises generating a multi-dimensional feature input based on the sensor data.

15. The processor-implemented method of claim 10, wherein blanking the sensor data comprises transmitting the sensor data to a paired device in connection with an indication that the sensor data is not to be used for determination of insulin dosages.

16. The processor-implemented method of claim 10, further comprising:
updating an outlier counter responsive to determining that the sensor data corresponds to an outlier measurement;
comparing a value of the outlier counter to a threshold; and
in response to determining that the value of the outlier counter exceeds the threshold, terminating a sensor device from which the sensor data was obtained.

17. The processor-implemented method of claim 10, further comprising:
after determining that the sensor data corresponds to the outlier measurement, determining whether the sensor data can be compensated;
in response to determining that the sensor data cannot be compensated, blanking the sensor data.

18. A processor-implemented method comprising:
obtaining sensor data;
applying, to the sensor data, a first machine learning model trained to identify outlier measurements;
determining, based on output from the machine learning model, that the sensor data corresponds to an outlier measurement;
in response to determining that the sensor data corresponds to the outlier measurement, applying, to the sensor data, a second machine learning model trained to identify whether the outlier measurement can be compensated;
determining, based on output from the second machine learning model, that the outlier measurement can be compensated; and
in response to determining that the outlier measurement can be compensated, applying a compensation to the sensor data to generate compensated sensor data.

19. The processor-implemented method of claim 18, wherein applying the compensation to the sensor data is instead of blanking the sensor data.

20. The processor-implemented method of claim 18, wherein the second machine learning model is configured to take, as input, a combination of in vivo sensor data obtained while a sensor from which the sensor data is in use by a patient and in vivo data obtained from the sensor or a sensor manufactured from a same substrate as the sensor.
